(19) United States
(12) Patent Application Publication
(10) Pub. No.: US 2011/0124049 A1
(43) Pub. Date: May 26, 2011

(54) INTEGRATED MICROFLUIDIC DEVICE FOR GENE SYNTHESIS

(76) Inventors: Mo-Huang Li, Singapore (SG); Jackie Y. Ying, Singapore (SG); Mo-Chao Huang, Singapore (SG); Sean Pin Ng, Singapore (SG)

(21) Appl. No.: 12/672,673

(22) PCT Filed: Jul. 31, 2008
(86) PCT No.: PCT/SG08/00282

§ 371 Date: Feb. 8, 2010

Related U.S. Application Data
(60) Provisional application No. 60/963,673, filed on Aug. 7, 2007.

(51) Int. Cl. C12P 19/34

(52) U.S. Cl. 435/91.2

(57) ABSTRACT

We report making an integrated micro-fluidic device for synthesizing double stranded DNA from short oligo-nucleotides. We demonstrate successful synthesis of a 760 bp gene segment from a pool of 39 oligonucleotides on a micro-fluidic device using both the one-step and two-step synthesis processes. We also describe purifying the double stranded DNA PCR product and filtering out sequence errors in the double stranded DNA product, all on the same device.

### REFERENCE TO RELATED APPLICATIONS

This application claims the benefit of U.S. Provisional Application No. 60/963,673, filed Aug. 7, 2007, the content of which is herein incorporated by reference.

### FIELD OF INVENTION

The invention relates to gene synthesis; specifically, to methods and systems for synthesizing double stranded DNA from short oligo-nucleotides directly on a microfluidic device.

### BACKGROUND OF THE INVENTION

Integrated microchip-based PCRs have been constructed using lab-on-a-chip technologies. These technologies and micro-PCR applications have been reviewed (Auroux et al, Lab Chip, 2004, 4, 534; Zhang et al, Biotech. Adv., 2006, 24, 243; Chenet al, Lab Chip, 2007, 7, 1413; Zhang and Xing, Nucleic Acids Res., 2007, 35, 4223).

Kong et al (Nucleic Acids Res., 2007, 35(8):e61, e-pub Apr. 2, 2007 and US Patent application publication US 2007/0281309) describes fabrication of a multi-chamber microfluidic device for de novo gene synthesis by assembling the precursor oligonucleotides and amplifying the assembled templates in a single reaction in the same chamber.

### SUMMARY OF THE INVENTION

We describe here an invention based in part, but is not limited to, our demonstration of an integrated microfluidic device capable of performing two-step gene synthesis to assemble a pool of oligonucleotides into genes with the desired coding sequence. The device comprised of two polymerase chain reactions (PCRs), temperature-controlled hydrogel valves, electromagnetic micromixer, shuttle micromixer, volume meters, and magnetic beads based solid-phase PCR purification, fabricated using a fast prototyping method without lithography process. The fabricated device is combined with a miniaturized thermal cycler to perform gene synthesis. Oligonucleotides were first assembled into genes by polymerase chain assembly (PCA), and the full-length gene was amplified by a second PCR. The synthesized gene was further separated from the PCR reaction mixture by the solid-phase PCR purification.

Accordingly, one aspect of our invention relates to a two-step method for synthesizing double-stranded DNA in a microfluidic device. In this aspect, the device comprises a PCR-assembly (PCA) chamber in controllable fluid communication with a polymerase chain reaction (PCR) chamber. The method comprises the steps of: (a) applying a time-varying thermal field to the PCA chamber containing a plurality of different oligonucleotides and polymerase, wherein each oligonucleotide has partial base complementarity with at least one other oligonucleotide, thereby assembling the oligonucleotides into templates for PCR in the absence of terminal PCR primers; (b) loading the templates produced in step (a) into the PCR chamber in the presence of a PCR precursor mix comprising the terminal PCR primers, dNTPs and polymerase; and (c) applying a time-varying thermal field to the PCR chamber, thereby obtaining a PCR product mixture comprising the double-stranded DNA.

Once the PCR amplication has concluded, the PCR product may be purified on the device. Accordingly, in the two-step method, the device may further comprise a purification chamber in controllable fluid communication with the PCR chamber. In this method there is the further step of: (d) loading the PCR product mixture into the purification chamber to immobilize the double-stranded DNA, thereby separating the double-stranded DNA from free dNTPs, primers and unpolymerized oligonucleotides. Immobilization may be effected by using magnetic beads. The double-stranded DNA may be extracted from the magnetic beads by subjecting the bead-immobilized DNA to heatshock conditions of 60° C. for 3 minutes.

An error-correcting step may be incorporated in the methods. When buffer conditions for carrying out error filtration are different from PCA or PCR conditions, then buffer exchange would be desired and the error filtration step would logically follow the purification step. Accordingly in the two-step method, the device may further comprise an error filtration chamber in controllable fluid communication with the purification chamber. In this method there is the further step of loading the purified double-stranded DNA into the error filter chamber to remove double-stranded DNA that contain base-pair mismatches.

The purification step may also be carried out before PCR amplification of the template. That is, the templates may be purified (and optionally, error-corrected) before being used for PCR. According, the device may further comprise a purification chamber in controllable fluid communication with the PCA chamber. This method comprises the step of loading the templates produced by PCA into the purification chamber to immobilize the templates, thereby separating the templates from free dNTPs and unpolymerized oligonucleotides; and then proceeding to PCR. Immobilization may be effected by using magnetic beads. The templates may be extracted from the magnetic beads by subjecting the bead-immobilized templates to heatshock conditions of 60° C. for 3 minutes.

As with the PCR products, the templates produced by PCA may also be subjected to error filtration. Accordingly, the device may further comprise an error filtration chamber in controllable fluid communication with the purification chamber. This method further comprises the step of loading the purified template into the error filter chamber to remove templates that contain base-pair mismatches; and then proceeding to PCR.

Another aspect of our invention relates to a one-step method for synthesizing double-stranded DNA in a microfluidic device in combination with a purification step. In this aspect, the device comprises a synthesis chamber in controllable fluid communication with a purification chamber, the method comprising the steps of:

(a) applying a time-varying thermal field to the synthesis chamber containing terminal PCR primers, polymerase, dNTPs and a plurality of different oligonucleotides wherein each oligonucleotide has partial base complementarity with at least one other oligonucleotide, thereby obtaining a PCR product mixture comprising the double-stranded DNA; and
(b) loading the PCR product mixture into the purification chamber to immobilize the double-stranded DNA, thereby separating the double-stranded DNA from free dNTPs, primers and unpolymerized oligonucleotides. Immobilization may be effected by using magnetic beads. The double-stranded DNA may be extracted from the magnetic beads by subjecting the bead-immobilized DNA to heatshock conditions of 60° C. for 3 minutes.

As with the PCR products and the templates in the two-step method, the device for one-step synthesis may comprise an error filtration chamber in controllable fluid communication with the purification chamber. In this method there is the further step of loading the purified double-stranded DNA into the error filter chamber to remove double-stranded DNA that contain base-pair mismatches.

The methods and devices as described herein may further comprise a micro-mixer to facilitate mixing of reaction components. Specifically, it may be helpful to mix the PCR precursor mix (dNPTs, polymerase and terminal PCR primers) with the templates produced by PCA. It may also be helpful to include a mixing step to optimize binding to the DNA-adsorbing solid phase media for purification.

The devices as described herein may be operably linked to a fluid-flow actuator, so that the flow of the fluids among the chambers is regulated. In certain embodiments, the fluid-flow actuator is a pump or a centrifuge. The fluids may move from one chamber to the next via channels comprising valves.

In certain embodiments, the valves are responsive to temperature changes. In particular, the valves that control sealing of the PCR chamber are preferably able to withstand at least 6.8 psi of pressure.

To carry out the synthesis reactions as described herein, the devices may be operably linked to a heating element, a cooling element, a temperature-sensor, and a temperature controller.

In another aspect, our invention relates to a microfluidic device for synthesizing double-stranded DNA, the device comprising a PCR-assembly (PCA) chamber configured to contain between 1 nL and 100 uL of fluid, a polymerase chain reaction (PCR) chamber configured to contain between 1 nL and 100 uL of fluid, and a chamber configured for solid-phase purification of the PCR product, wherein the chambers are in controllable fluid communication with one another.

The microfluidic device may further comprise a mixing chamber configured to mix products of the PCA reaction with a PCR reaction mix.

The microfluidic device may further comprise a plurality of different oligonucleotides in the PCA chamber, each oligonucleotide having partial base complementarity with at least one other oligonucleotide.

The microfluidic device may further comprise a chamber configured for error filtering of the PCR product.

In certain embodiments of the microfluidic device, the chambers are in controllable fluid communication with one another via channels comprising valves. The valves may be responsive to temperature changes and those valves that control sealing of the PCR chamber are, in some embodiments, able to withstand at least 6.8 psi of pressure.

The microfluidic device may be operably linked to a fluid-flow actuator.

In another aspect, our invention relates to a system for synthesizing double-stranded DNA, the system comprising the microfluidic device as described herein, operably linked, to a heating element, a cooling element, a temperature-sensor, and a temperature controller.

### BRIEF DESCRIPTION OF THE DRAWINGS

FIG. 1. (A) Schematic of one embodiment of the gene assembly fabrication process. (B) Schematic illustration of embodiments of PCR-based gene synthesis. One-step synthesis combines PCA and PCR amplification into a single stage. The two-step synthesis is performed with separate stages for assembly and amplification.

FIG. 2. Embodiments of (A) PCA chip; (B) Schematic of the thermal cycler; (C) Photograph of the thermal cycler with PCA chip.

FIG. 3. Embodiment of integrated two-step gene synthesis chip. (A) Schematic; and (B) photograph of the microfluidic device (40 mm×35 mm).

FIG. 4. Embodiments of (A) Schematic; and (B) photograph of DNA extraction/buffer exchange chip with metering chambers (M1 and M2), inlet (A1) and outlet (A2) for loading wash and elution buffers, beads chamber (C1), and product collection chamber (C2).

FIG. 5. Embodiments shown as (A) Printed mold of photosensitive resin for single-chamber chip. (B) Fabricated single-chamber chip with hydrogel valves. The PCR reactions and hydrogel valves were controlled by two separate thermoelectric heaters (TE 1 and TE 2). (C) Photograph of a two-step gene synthesis chip with solid-phase PCR purification (65 mm×50 mm).

FIG. 6. Schematic of embodiments of the MutS error filtering steps. (A) Conventional method using gel electrophoresis to separate the mismatched DNAs captured by MutS enzyme from the matched DNA with correct sequence; (B) Solid-phase MutS error filter. MutS enzyme is immobilized on magnetic particles or nickel chelate particles.

FIG. 7. DNA quantity (▪) before and (•) after MutS error filtering. (A) 760 bp DNA sample with a mixture of 50% incorrect sequence (2 deletions and 2 insertions) and 50% correct sequence; (B) 760 bp DNA sample with 100% perfect sequence.

FIG. 8. Embodiments of (A) Chip-based gene synthesis system comprising PCA assembly, PCR amplification, buffer exchange and error filtering; (B) Schematic of fully integrated device for gene synthesis.

FIG. 9. Conceptual description of gene assembly fabrication process sequence.

FIG. 10. Schematic of the bioinformatic software for breaking the DNA sequence into optimal oligonucleotides.

FIG. 11. One embodiment for PCA. The methods of overlap extension, successive extension, and thermodynamic balanced inside-out polymerase cycling gene assembly (PCA), and ligation based gene synthesis.

FIG. 12. Embodiments of (A) The Gene-CD instrument; (B) Schematic illustration of the microfluidic structure employed for the Gene-CD platform.

FIG. 13. Schematic diagrams of device operations in one embodiment. (a) Oligonucleotides and PCR mixture were loaded into PCA chamber. PCA was then conducted. (b) PCA-assembled solution was mixed with fresh PCR mixture containing outer primers. (c) Mixed reagent was positioned in PCR chamber, and the PCR amplification was performed. (d) PCR-synthesized product and ChargeSwitch reagent were pumped and loaded into beads chamber. Magnetic beads were captured by a magnet. (e) Magnetic beads were washed. (f) Elution buffer was loaded and mixed with magnetic beads. Synthesis product was eluted into elution buffer.

FIG. 14. (a) Photographs of one micromixer embodiment. Colored dyes (blue and red) were well mixed after being shuttled three times between two chambers. (b) Schematic illustration of the experimental arrangement with a syringe pump, electromagnetic mixer, thermoelectric heaters and data acquisition.

FIG. 15. The thermal response of in situ photopolymerized hydrogel valve. The valve functions were highly repeatable. The insets showed the transitions of valve functions.

FIG. 16. Thermal cycling profiles of the custom-built PCR thermal cycler. A thermocouple mounted on the heater was used in the temperature feedback control (heater temperature) for thermal cycling. The temperature difference between the heater surface and within the PCR chamber (chamber temperature) was compensated using a LabVIEW program.

FIG. 17. (A) PCA results of the commercial thermal cycler and the single-chamber device. Different oligo concentrations are used to optimize the PCA recipe. (B) Two-step gene synthesis results of the commercial thermal cycler and the integrated two-step gene synthesis device.

FIG. 18. Agarose gel (1.5%) electrophoresis showing the synthesis yields with oligonucleotide concentrations of 5-25 nM and outer primer concentrations of 0.1-0.4 μM for the two-step process. Syntheses were conducted using a commercial thermal cycler. (a) PCA results. (b) PCR amplification results.

FIG. 19. Agarose gel (1.5%) electrophoresis comparing the synthesis results conducted within commercial thermal cycler (machine) and microfluidic device. (a) One-step process (device: single-chamber chip); and (b) two-step process (device: two-step chip) conducted with an oligonucleotide concentration of 10 nM and a primer concentration of 0.4 μM.

FIG. 20. The effect of elution temperature and incubation time on DNA extraction conducted within microfluidic device (▪: 3 min) and standard PCR tube (□: 3 min; ⋄: 2 min).

### DETAILED DESCRIPTION OF EMBODIMENTS

Our aim is to develop an integrated lab-on-a-chip microsystem to perform automatic gene assembly from short synthetic oligonucleotides. The oligonucleotides are assembled into a DNA sequence for encoding genes and genomes based on known assembly methods including polymerase chain reaction (PCR) and ligase chain reaction (LCR). Components required for performing gene assembly are developed, miniaturized, and integrated into on a microfluidic device for gene assembly. We describe the invention in terms of their components and elements and illustrate the invention in terms of its various embodiments. The invention should not be limited to the specific embodiments exemplified or to the explicit combinations of elements described herein.

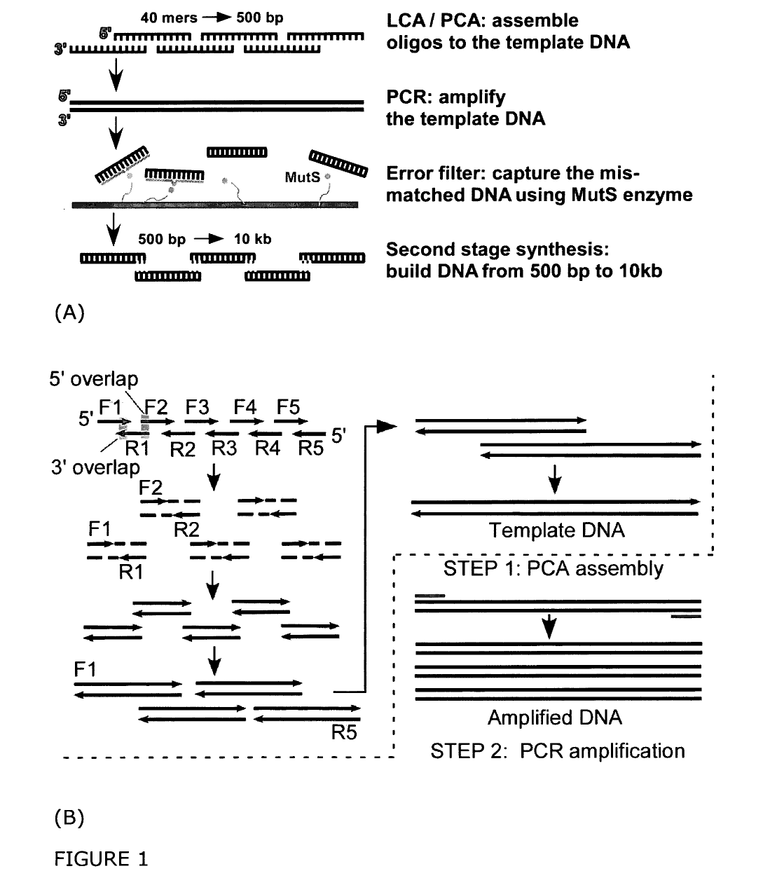

FIG. 1(A) shows the concept in one embodiment of the two-step overlapping gene assembly method for creating a synthetic gene. This embodiment includes four process steps, which are polymerase chain assembly (PCA), polymerase chain reaction (PCR) amplification, buffer exchange (not shown in FIG. 1(A)), and error filtering. The PCA step assembles a pool of short oligonucleotides (with a length of 20-60 bases long) into long double-strand DNA (called template) with the desired length and sequence information. The quantity of the assembled template DNA is then amplified by the PCR step (FIG. 1B). As the assembled product also contains some DNA with incorrect sequence, the product is filtered by using an enzymatic error filter containing MutS enzyme. This step purifies the assembled product. To integrate these steps into a chip, an extra step (buffer exchange) is added, in conjunction with the PCR amplification and error filtering steps. This step will extract the full-length template, and release the full-length template to a buffer optimized for error filtering.

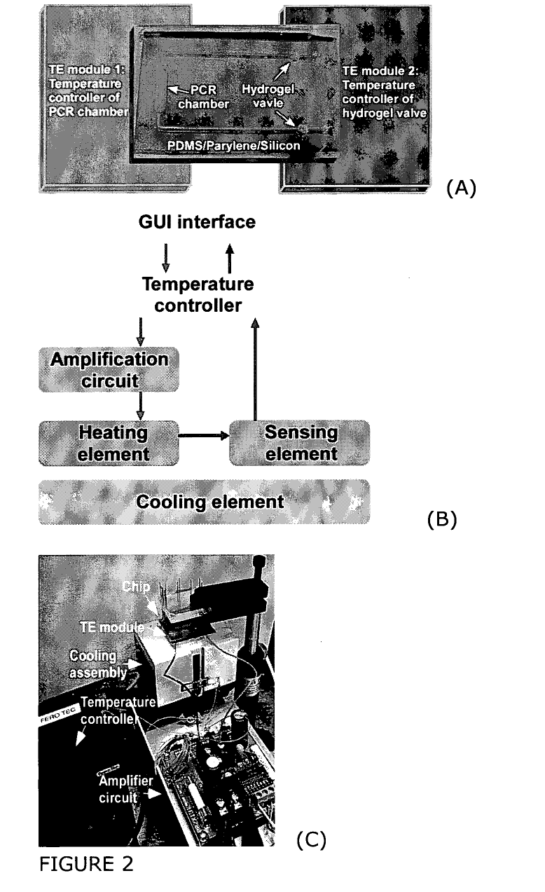

Gene synthesis on a microfluidic device (also called a “chip”) may be composed of a number of components. FIG. 2(A) illustrates the miniaturized thermal cycler for performing PCA or PCR. One design is composed of a PDMS fluidic structure on a silicon substrate with photo-polymerized hydrogel valves. The hydrogel valves are used to seal the PCA or PCR reagents, and to prevent the reagent evaporation during temperature cycling. The function of this chip is controlled by two thermoelectric modules: one for thermal cycling and another for controlling the hydrogel valves. The schematic and the actual set-up of the thermal cycler are shown in FIGS. 2(B) and (C), respectively. The entire system may be controlled by LabView software.

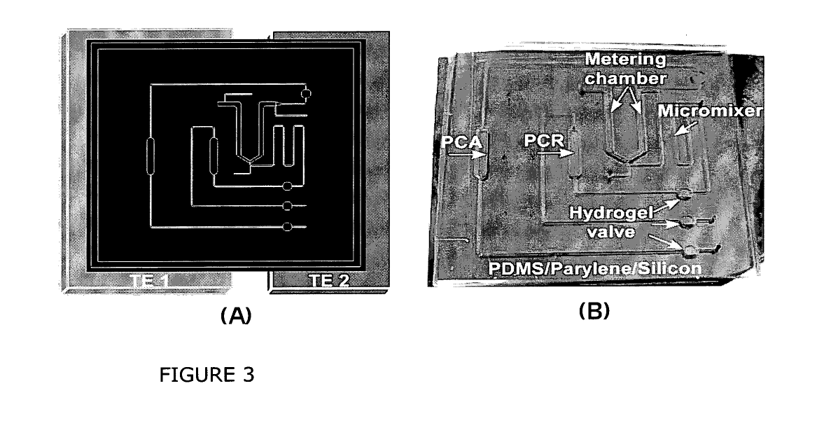

FIG. 3 shows one design of an integrated two-step gene synthesis chip. This chip integrates the PCA and PCR steps in the same chip with other microfluidic components for reagent volume metering and mixing. It can perform the PCA assembly, followed by the PCR amplification using the same thermal cycler described in FIG. 2(C).

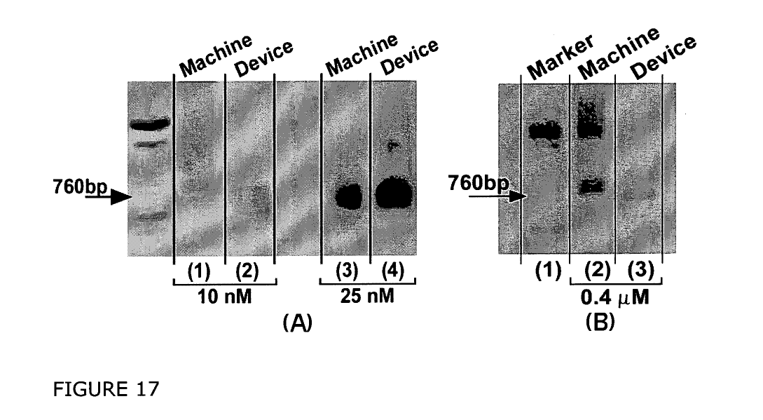

We demonstrated the performance of the fabricated device by assembling 760 bp DNA (a segment of GFPuv) from a pool of short oligonucleotides (40 bases). The gene synthesis process was first optimized on a single chamber thermal cycling chip (FIG. 2(A)). Then the optimized recipe was used to synthesize 760 bp DNA on an integrated two-step gene synthesis chip (FIG. 3). The synthesis result obtained with the chip is compared with that of a commercial thermal cycler (FIG. 17). The single-chamber device successfully generates the full-length product from oligonucleotides with a concentration as low as 10 nM (FIG. 17(A)). It also produces a larger quantity of full-length DNA than the commercial thermal cycler. FIG. 17(B) shows the results from the two-step integrated chip. The clear band in the gel results from our device illustrates that most of the synthesized product is full-length DNA (FIG. 17(B)).

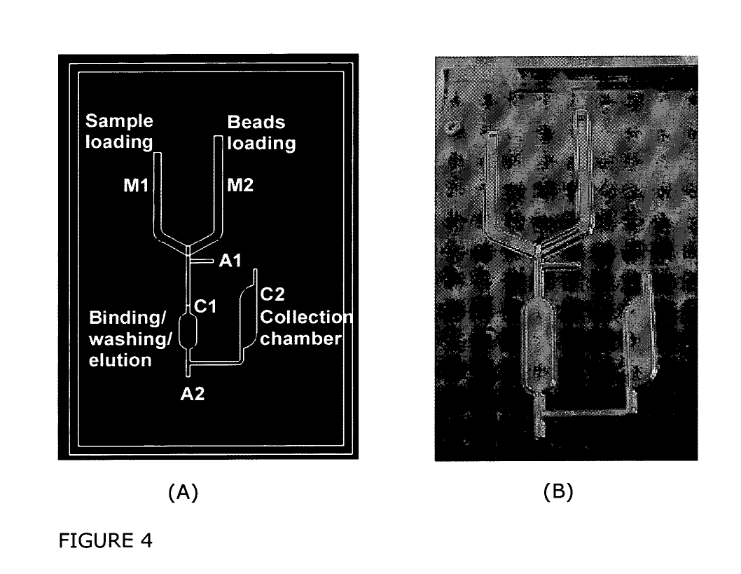

After PCR amplification, the sample may be subjected to DNA extraction and buffer exchange. The former process aims to extract the full-length DNA from a pool of assembled products that contain DNAs of various lengths. One way to do this is by integrating the solid-phase DNA extraction process in a chip. The basic concept is to use silica-coated magnetic beads to capture long DNA at a low pH value, wash the beads with the wash buffer, and then release the captured DNA into another buffer with a pH value of 8.5. FIG. 4(A) shows the schematic of such a DNA extraction device. The magnetic beads are confined in the beads chamber (C1) using a tiny magnet located underneath the beads chamber. The DNA extraction chip can be integrated with the PCR chip by connecting the outlet of the PCR chip to the sample loading inlet at FIG. 4(A). The fabricated DNA extraction chip is shown in FIG. 4(B). To demonstrate the concept, we have employed silica-coated magnetic beads from Invitrogen. The silica-coated magnetic beads from other commercial suppliers or IBN can also be used. We have optimized the process parameters of this DNA extraction chip by using 100 bp DNA ladder as sample. The yield, defined as the percentage of DNA captured and released, is 42% for our chip using the standard protocol provided by Invitrogen. By incorporating our own release process (which is enhanced by heating), we have increased the yield to 70%. In short, we have successfully developed the DNA extraction chip, which can be integrated with the PCR chip.

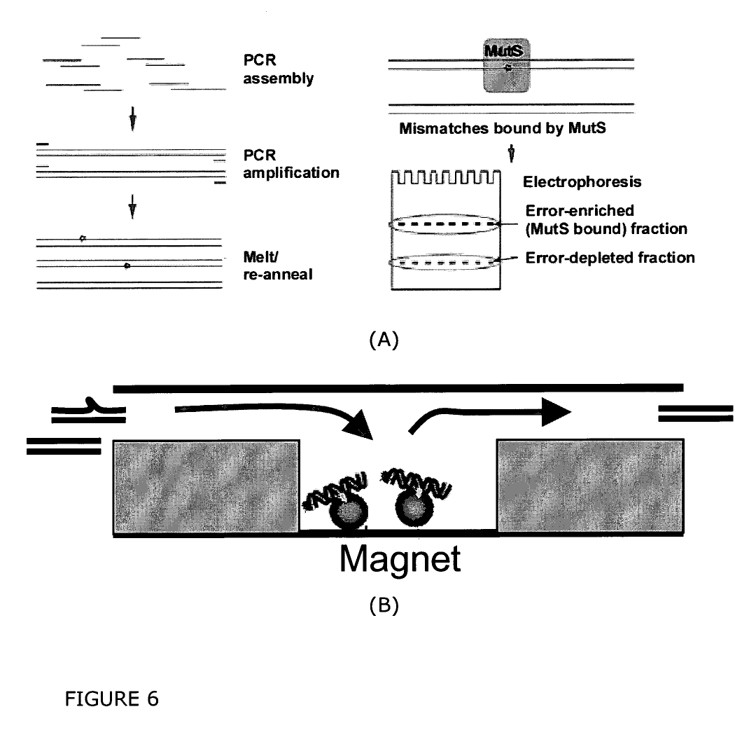

One desirable chip component for gene synthesis is the error filter. The assembled DNA product contains DNAs with correct and incorrect sequences. One way to remove the erroneous DNAs from the product, the enzymatic error filter is used with MutS enzyme (see FIG. 6). The MutS enzyme is capable of recognizing the DNAs with mismatch and binding to the mismatched site, but does not affect the correct DNA. The conventional method uses the gel electrophoresis to separate the correct DNA from the MutS-bound DNA (in solution) (FIG. 6(A)). We have used a solid-phase error filter with the MutS enzyme immobilized on magnetic beads (FIG. 6(B)). The mismatched DNAs are captured by the immobilized MutS enzyme, and the correct DNA would just flow through the filter unaffected.

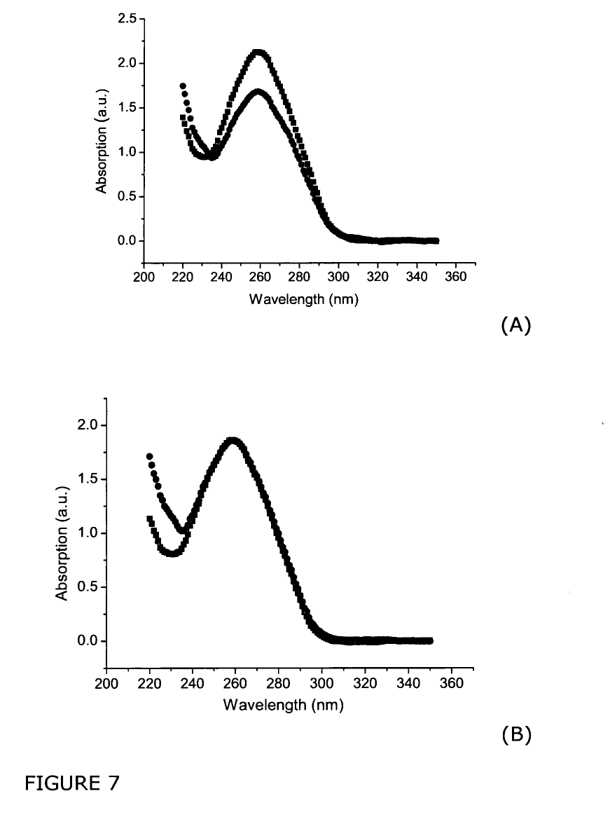

We have demonstrated this solid-phase error filter by using M2B2 (Genecheck), which are magnetic beads with immobilized MutS enzymes. The experiment was performed using 50 μL vials for samples with 50% mismatched sequence (FIG. 7(A)) and with 100% perfect sequence (FIG. 7(B)). The MutS error filter successfully removed the erroneous DNAs from the product. As the solid-phase error filter also uses magnetic beads, the chip design is similar to the solid-phase DNA extraction chip. Moreover, the solid-phase error filter can be easily integrated with other components. We contemplate using His-tagged MutS on nickel chelate particles as an immobilization method which will replace the commercial M2B2. This would not affect the process and chip design of the solid-phase error filter:

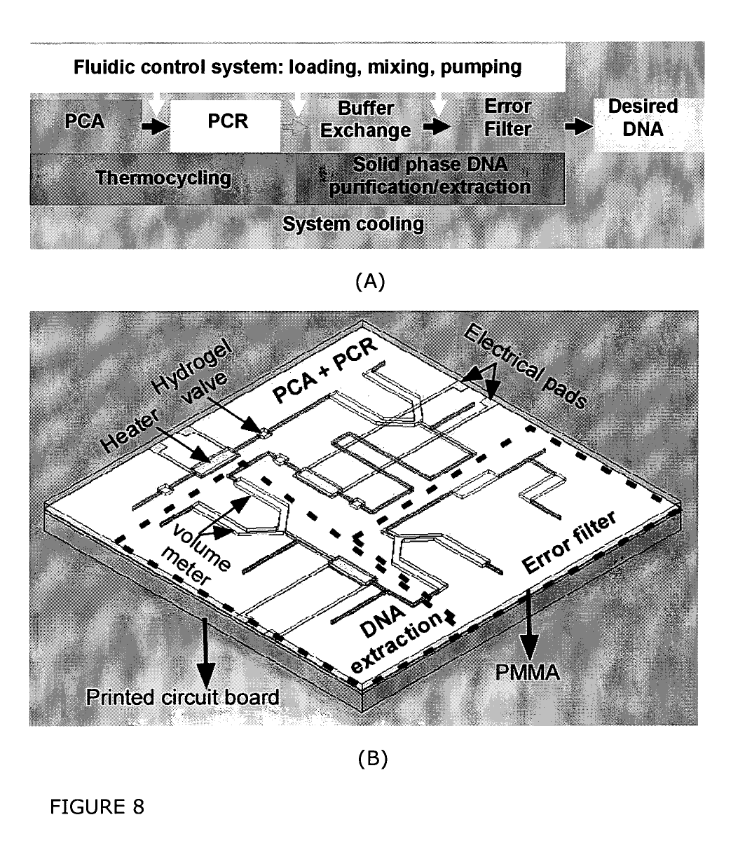

We have successfully developed components that enable automatic gene synthesis on a chip. Beside the chip design, we have also developed hardware and software for reagents regulation, temperature cycling, solid-phase purification and error filtering. FIG. 8(A) shows the schematic diagram of the system. In one embodiment, our invention includes the following:

1. Device components for PCA, PCR, solid-phase DNA extraction, and solid-phase error filter. We have demonstrated the performance of these components on separate microfluidic chips. These components can be integrated into a single chip and become a microsystem that is capable of performing gene synthesis automatically.
2. Novel methods for performing gene synthesis on a chip. We have replaced the conventional gel electrophoresis method for DNA extraction and error filtering by solid-phase methods with fluidic regulation controlled by hydrogel valves. The yield of DNA extraction is also enhanced by incorporating a heating step.
3. The results indicate that our device can outperform conventional methods. Our device provides higher efficiency and quantity of assembled product than conventional methods where gene synthesis is conducted using manual pipetting, commercial thermal cycler for PCA and PCR, and gel electrophoresis for buffer exchange and error filtering.

We present a schematic of the integrated chip in FIG. 8(B). The device consists of a printed circuit board (PCR, bottom layer) with integrated thin film heaters and temperature sensors. The thin film heater and temperature sensors located beneath the PCA and PCR chambers are for thermal cycling. The thin film heater beneath the DNA extraction chamber is to increase the release yield. The hydrogel valve can also be controlled by an integrated heater. The chip can be powered by an electrical module through the electrical connection pads around the chip. The integrated chip may employ polymethyl-methacrylate (PMMA) (top layer) to create the microfluidic structure.

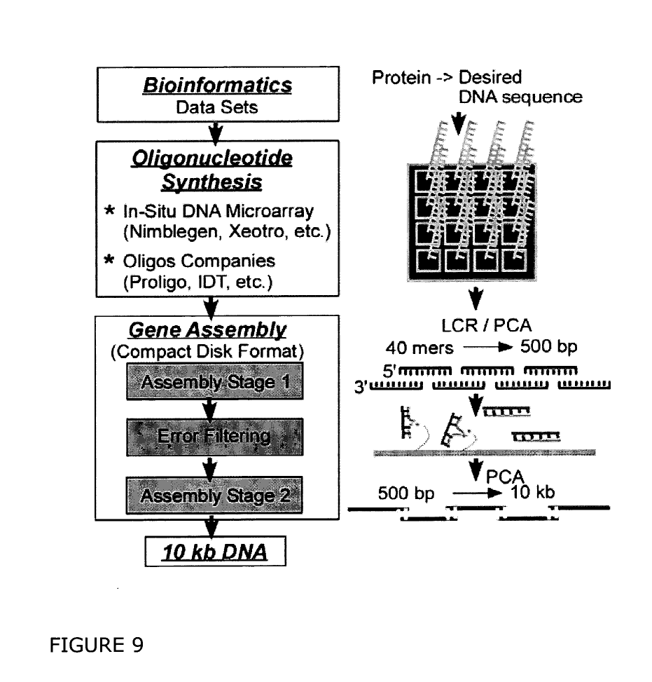

FIG. 9 shows design, synthesis and assembly of a gene using overlapping oligos. It includes the processes of bioinformatics, oligonucleotide synthesis, and gene assembly where the bioinformatics partition the DNA sequence into optimal short oligomers. Then, these oligomers are synthesized either using in-situ DNA microarray (Richmond et al, Nucleic. Acids Research, 3, 5011-8, 2004) or commercial oligonucleotide synthesizers. These oligomers are then released, purified, and assembled into longer DNA segments using multi-steps ligation and polymerase cycling assembly (PCA) methods. DNA segments with incorrect sequence are removed by an error filtering before final PCR amplification (Carr et al, Nucleic Acids Research, 32(20), e162, 2004).

In our demonstrated embodiments, the PDMS/silicon chip was fabricated by utilizing printed three-dimensional mold of photopolymerized resin. The protein adsorption and PCR mixture evaporation in PDMS were eliminated by coating the device with a thin layer of parylene. The fluidic control was realized with a precision syringe pump, and thermally activated hydrogel valves. PCR reaction mixture was sealed during thermal cycling by in situ hydrogel valves, which were tested and capable of withstanding pressures of ≧8 psi without visible leakage.

We showed that microfluidic syntheses were successfully attained with low oligonucleotide concentration of 10 nM and primer concentration of 0.4 μM using one-step and two-step PCR-based gene synthesis processes. More full-length products were generated by the two-step process, but the resulting error rates of both processes were not very different. The synthesized products were verified by DNA sequencing to have an error rate of ˜1 per 250 bases, comparable to the control experiments conducted in PCR tube with a commercial thermal cycler.

We have successfully used this device to synthesize a green fluorescent protein fragment (GFPuv) (760 bp), and obtained comparable synthesis yield and error rate with experiments conducted in PCR tube within a commercial thermal cycler. The resulting error rate determined by DNA sequencing was 1 per 250 bp. To our knowledge, this is the first microfluidic device demonstrating integrated two-step gene synthesis.

Along with the integrated gene synthesis chip, we describe a microfluidic design to purify the synthesis product and prepare buffer solution for downstream application. We demonstrate using silica-coated magnetic beads for the solid-phase PCR purification to separate the synthesized product from the PCR reaction mixture. On testing, we found that a short heat shock can be used to enhance the DNA extraction efficiency. We showed that a 70% extraction efficiency and microgram-level DNA loading capacity were obtained by applying a short heat shock (e.g. 60° C. for 3 min) before DNA elution. This would help prepare the synthesized gene in a suitable buffer solution for in vitro cell-free protein synthesis, or integrate DNA error correction methods on chip to improve the accuracy of synthesized products. For the embodiment we demonstrated, the process takes ˜2 hrs including two PCRs (30 cycles each) and the PCR purification (<10 min), producing ˜2 μg DNA products (752 bp).

While this work was demonstrated with fluidic design in microliter scale to direct compare with experiments in PCR tube, the volume of reactors and structures' dimensions can be scaled down substantially to provide more cost-effective gene synthesis. The design of hydrogel valves, reaction chambers, micromixers, and PCR purification are flexible and can be scaled down without significant design modification.

### Oligo Design for DNA Assembly

Gene sequence fidelity and production efficiency depend on specificity and completeness of building-block oligos hybridization. The primary bioinformatics objectives are to ensure that each oligomer has one and only one complementary target sequence and to ensure that each oligomer is free of any secondary structure that would preclude gene assembly. Thus it is best to break down a complete gene (2 kb to 10 kb) into assembly sequences such that each of the sequences is unique and structure free.

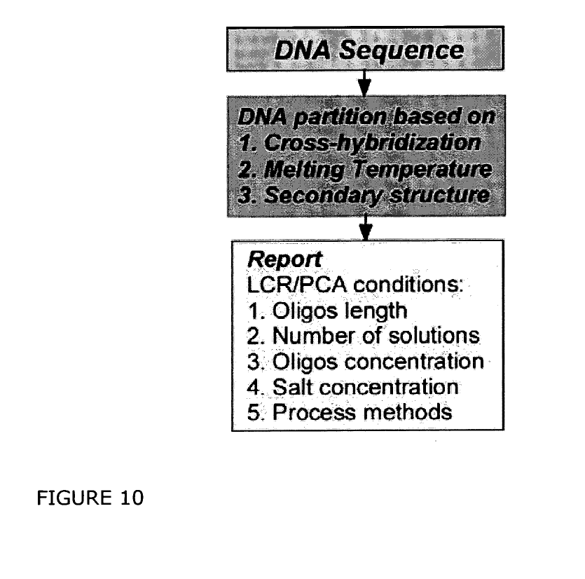

The guidelines for partitioning DNA sequence into optimal oligos (FIG. 10) are similar to that of selection of optimal DNA oligos for DNA microarray among various genes, where the oligos are desired to have an uniform melting temperature (Tm), no cross-hybridization and secondary structure to form hairpins or dimers. Therefore, the algorithms and methods developed for oligos microarray (Li and Stormo, Bioinformatics, 17(11), 1067-76, 2001; Chou et al, Bioinformatics, 20(17), 2893-2902, 2004) are adapted to develop high performance bioinformatics for gene assembly.

The DNA sequence can be considered as a text made of four letters (A, T, G, and C) which contains string information such as keyword (cross-hybridization) and duplication (repeats or high G+C contents). By first identifying the problematic sequence regions, we believe this approach provides several advantages over the current gene assembly software DNAWorks (Hoover and Lubkowski, 30(10), Nucleic Acids Research, pp. e43, 2002) and Gene2Oligo (Rouillard et al, Nucleic Acids Research, 32, Web Server issue, w176-180, 2004). First, the troublesome DNA segments can be predicted and circumvented. DNA containing repeated regions or high G+C content will hinder the gene assembly, which can be predicted using sequence landscape algorithms (Li and Stormo, Bioinformatics, 17(11), 1067-76, 2001). The sequence is then divided into segments; a set of oligonucleotides is designed for each segment; these are assembled in parallel. Finally all the synthesized segments are combined into the final sequence. In combining the segments, these are used in place of short oligonucleotides to assemble into a template for PCR. Multiple rounds of assembly and synthesis reactions may be required depending on the number of oligonucleotides and fragments are required to form the final desired sequence. Second, the cross-hybridization problem can be considered as keyword searching problem with algorithms well-developed in computer science to improve efficiency compared to commonly used BLAST program. Third, the trend of self-complementary can be predicted using string suffix array algorithms (Li and Stormo, Bioinformatics, 17(11), 1067-76, 2001; Chou et al, Bioinformatics, 20(17), 2893-2902, 2004) to avoid unnecessary computation without using Mfold program to calculate the free energy of secondary structure.

Optimal oligonucleotides are designed for gene assembly by LCR or PCA based on the approach described. The long DNA sequence is first divided into segments with length of 500 bp-1 kbp, and then each segment is partitioned into optimal oligos based on length priority or melting temperature priority methods. For length priority method, all the oligos have the same length defined by the user or calculated from the user-defined melting temperature (Tm) and acceptable melting temperature variation (ΔTm) among the oligo set. For melting temperature priority method, the oligos are allowed having different lengths in order to obtain a uniform Tm among the oligo set. This method provides an advantage on reducing synthesis errors due to the mis-hybridization of mutated oligos. By having a uniform Tm across the oligo set, one can perform LCR or PCA at a temperature closer to the mean Tm, which will diminish the probability of incorporation of the mutated oligos with lower Tm into a final product.

### Microfluidic Device

Microfluidic devices of the present invention are silicon-based chips and fabricated using a variety of techniques, including, but not limited to, hot embossing, molding of elastomers, injection molding, LIGA, soft lithography, silicon fabrication and related thin film processing techniques, photolithography and reactive ion etching techniques. In one embodiment, glass etching and diffusion bonding of fused silica substrates may be used to prepare microfluidic chips. The microarchitecture of laminated and molded microfluidic devices can differ.

The University of Pennsylvania (U.S. Pat. Nos. 5,498,392; 5,587,128; 5,955,029; 6,953,675) disclosed microfabricated silicon-based devices for performing PCR. The patent disclosed small, mass produced, typically one-use, disposable “chips” for rapid amplification of cellular or microbial nucleic acids in a sample. The device included a sample inlet port, a “mesoscale” flow system, and a means for controlling temperature in one or more reaction chambers. Off-chip pumps were used to control fluid flow and to deliver reagents. Heating and cooling means disclosed included electrical resistors, lasers, and cold sinks. Printed circuits, sensors on the chip, and pre-analytical binding means for trapping and concentrating analyte were disclosed. A common fluid channel was used to transport cell lysis waste to an open vent or to an off-chip site. Analytical devices having chambers and flow passages with at least one cross-sectional dimension on the order of 0.1 μm to 500 μm were disclosed. Reaction volumes of 5 μL or lower were predicted.

Design of microfluidic systems using laminate technology allows multichannel analysis and the formation of 3-dimensional microfluidic systems of varying degrees of complexity (Jandik et al. J. Chromatography A, 954: 33-40, 2002; Cabrera. “Microfluidic Electrochemical Flow Cells: Design, Fabrication, and Characterization”, Thesis, 2002 Department of Bioengineering. Seattle, University of Washington; Cabrera et al. Analytical Chemistry, 73:658-666, 2001; McDonald and Whitesides. Accounts of Chemical Research, 35(7), 491-499, 2002; and McDonald et al. Electrophoresis, 21, 27-40, 2000; all of which are incorporated herein in their entirety, to the extent not inconsistent herewith).

Suitable materials for fabricating a microfluidic device include, but are not limited to, cyclic olefin copolymer (COC), polycarbonate, poly(dimethylsiloxane) (PDMS), poly(methyl methacrylate) (PMMA), glass.

Due to the hydrophobic nature of polymers such as PDMS, which adsorbs proteins and inhibits certain biological processes, a passivating agent may be necessary (Shoffner et al. Nucleic Acids Research, 24:375-379, 1996). Suitable passivating agents are known in the art and include parylene and DDM. See Zhang et al, Biotech. Adv., 2006, 24, 243 for a description of microfluidic device fabrication technology, including materials, design and surface passivation techniques for PCR microfluidics.

‘Controllable fluid communication’ refers to the device design such that the reaction fluids can be moved from one location to another within the device in a manner regulated by the user.

Once way to provice communication between the device chambers is via channels. One form of a microfluidic channel is a fluid channel having variable length. In some embodiments, one dimension of the cross-section of a channel is less than 500 μm. In other embodiments, one dimension of the cross section of a channel is larger than 500 μm. Microfluidic fluid flow behavior in a microfluidic channel is non-ideal and may be dependent on wall wetting properties, roughness, liquid viscosity, surface tension, adhesion, and cohesion. Further, flow in channels based on rectangular or circular cross-sectional profiles are controlled by the diagonal width or diameter. The most narrow dimension of a channel has the most profound effect on flow. Vias in a channel can be designed to promote directional flow, a sort of solid state check valve.

The microfluidic device of the present invention may comprise a PCA chamber and a PCR chamber (2-step chip) or the two reactions may take place within the same chamber (‘synthesis chamber’; 1-step chip). Channels are one way to connect the chambers. The volume in each chamber may range from 1 nL to 100 μL. Specifically, the volume of each chamber may be between 5 nL and 10 nL; 10 nL and 50 nL; 50 nL and 1 μL; 1 μL and 10 μL; 7 μL and 20 μL; 10 μL and 20 μL; 20 μL and 40 μL; 40 μL and 70 μL; and 70 μL and 100 μL.

One way to control fluid access to a chamber on the device is by providing valves. Microfluidic valves include for example hydraulic, mechanic, pneumatic, magnetic, and electrostatic actuator flow controllers with at least one dimension smaller than 500 μm. A representative flap valve of the genus is described in U.S. Pat. No. 6,431,212. Also included are hydrogel valves, pinch valves, wax valve, membrance valves, check valves and elastomeric valves. Patents describing species of microfluidic valves include U.S. Pat. Nos. 5,971,355; 6,418,968; 6,620,273; 6,748,975; 6,767,194; 6,901,949; and 6,802,342.

Controlling the flow of fluid through channels may occur through the use of flow control mechanisms that include an expandable material. Such flow control mechanisms may be formed as part of the device and may include, for example, materials that swell upon contact with a fluid, such as, for example, water, a solvent, or the like. Examples of suitable materials for this use include hydrogels, polymers (e.g., swellable polymers), such as, for example, polyacrylamide, expandable materials commonly referred to as superabsorbent polymers (SAPs), and/or other available materials. Hydrogels may swell or collapse in response to a number of factors, for example, pH, temperature, ionic strength of a solution, or any combination thereof. By swelling or collapsing, the hydrogel may regulate the flow of fluid through a channel. For example, hydrogel may swell at temperatures below 32° C. thus blocking flow through a channel and shrink at a temperature above 32° C., thus allowing flow through the channel.

Reversible blocking may be used to perform serialized reaction processes within a microfluidic device. For serialized reaction processes, it may be desirable to sequence a series of chemical reactions and/or processes within a microfluidic device without exposing the reaction materials to the environment once they have been introduced into the microfluidic device.

Common materials for hydrogels include e.g. poly-NIPAAm, polyvinyl alcohol, sodium polyacrylate, acrylate polymers and copolymers with an abundance of hydrophilic groups. Natural hydrogel materials include agarose, methylcellulose, hylaronan, and other naturally derived polymers.

A microfluidic device may include a mixing module such as a shuttle mixer or an electromagnetic mixer. In shuttle mixing, a solution is shuttle between two chambers connected by a narrow channel. The abrupt opening at the channel-chamber junctions to create chaotic advection at the junctions and recirculates the flow. In electromagnetic mixing, alternating magnetic forces were generated to agitate magnetic beads in the microfluidic device, thus agitating the solution containing the magnetic beads. In another aspect, two liquid streams are made to flow through a channel such that the liquids are mixed during their residence time in the channel. For a given velocity of the fluid, the residence time of the liquid is increased, by increasing the length of the channel so as to ensure complete mixing. In another aspect, the mixer channel is branched into multiple narrower channels so as to ensure mixing in a shorter residence time.

Inlets comprise openings into a microfluidic channel. Inlets can fluidically connect a microfluidic channel to tributary microchannels, which are branching channels off of a main microfluidic channel, and may also be connected to valves, tubes, syringes, and/or pumps for the introduction of fluid into the microfluidic device. Outlets comprise openings out of a microfluidic channel and can also be connected to collection ports, absorbent material for removing fluid from the outlet.

It is sometimes necessary to control the volume of fluid volumes within the microfluidic device. A volume meter may comprise a chamber or a channel of defined volume at demarcated sections such that introduction of fluid to the demarcated sections result in a defined volume of fluid.

A fluid flow actuator allows directional movement of fluids within a microfluidic device. Exemplary actuators include syringe pumps, bulbs, bellows, diaphragms, or bubbles intended to force movement of fluids, where the substructures of the pump have a thickness or other dimension of less than 1 millimeter. Such pumps include the mechanically actuated recirculating pumps described in U.S. Pat. No. 6,743,399 and U.S. application publication 2005/0106066. Microfluidic pumps may be operated by hand or by robotics. Electroosmotic pumps are may also be used to regulate the flow of fluid in microfluidic devices.

Alternatively, centrifugal force is used to propel fluid in the microfluidic device. U.S. Pat. No. 5,610,074 described a centrifugal rotor for the isolation, in a sequence of steps, of a substance from a mixture of substances dissolved, suspended or dispersed in a sample liquid. Multiple samples are processed simultaneously by means of a plurality of fractionation cells, each of which contains a series of interconnected, chambered and vented compartments in which individual steps of the fractionation and isolation procedure take place. In this centrifugal rotor, the specific compartment occupied by the sample liquid or one of its fractions at any stage of the process is governed by a combination both the speed and direction of rotation of the rotor and gravitational force. The interconnections, chambers and passages of each compartment are sized and angled to prevent predetermined amounts of sample and reagent liquids from overflowing the compartment.

### Gene Assembly and Synthesis

Genes or genomes have been synthesized de novo from oligonucleotides to assemble a viral genome (7.5 kb; Cello et al, Science, 2002, 297, 1016), bacteriophage genome (5.4 kb; Smith et al, Proc. Natl. Acad. Sci. USA, 2003, 100, 15440), and a gene cluster as large as 32 kb (Kodumal et al, Proc. Natl. Acad. Sci. USA, 2004, 101, 15573). The longest synthetic DNA reported to date is 582 kb, the genome of a bacterium (Mycoplasma genitalium) by Venter and co-workers (Gibson et al, Science, 2008, 319, 1215). Furthermore, DNA synthesis has been successfully combined with high-density DNA microarray technologies (Tian et al, Nature, 2004, 432, 1050 and Richmond et al., Nucleic Acids Res., 2004, 32, 5011) providing millions of unique oligonucleotides at a significantly lower cost (on the order of 1 cent per oligonucleotide) compared to the conventionally synthesized oligonucleotides (USD 0.2 per base). DNA biomolecule as large as 15 kb (Tian et al, Nature, 2004, 432, 1050) has been successfully constructed with oligonucleotides from DNA microarray thus far.

Gene synthesis using a pool of 600 distinct oligonucleotides have been demonstrated (Tian et al. Nature, 432:1050, 2004). However, with increasing complexity of oligonucleotide pools, synthesis may become unfeasible. Assuming that a pool of 600 distinct oligonucleotides is the pool limit, and that each oligonucleotide is on average 40 bp with 20 bp overlap, then the largest possible double stranded DNA fragment produced is about 1.2 kb in length. As a result, for genes and DNA fragments larger than about 1.2 kb, more than one rounds of synthesis on a chip may be required. PCR assembly and synthesis from oligonucleotides as disclosed herein may be adapted for use in series, as described below. We envision that a single round of PCR assembly and synthesis from oligonucleotides in a microfluidic device would be routine for producing DNA of up to 1.5 kb; specifically in ranges from 300 bp to 1.2 kb and 500 bp to 800 bp. We envision that it would be practical to use a chip to make DNA of at least about 100 bp.

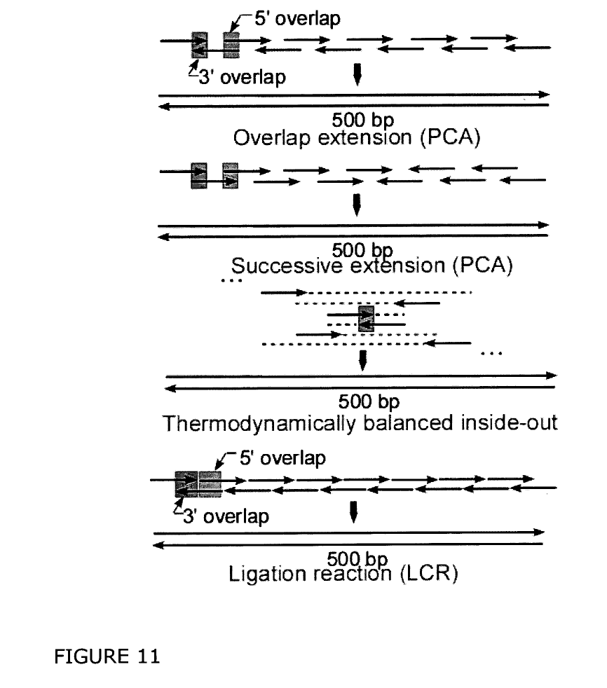

A variety of gene assembly methods exist, ranging from methods such as ligase-chain reaction (LCR) (Chalmers and Curnow, Biotechniques, 30(2), 249-52, 2001; Wosnick et al, Gene, 60(1), 115-27, 1987) to suites of PCR strategies (Stemmer et al, 164, Gene, 49-53, 1995; Prodromou and L. H. Pearl, 5(8), Protein Engineering, 827-9, 1992; Sandhu et al, 12(1), BioTechniques, 14-6, 1992; Young and Dong, Nucleic Acids Research, 32(7), e59, 2004; Gao et al, Nucleic Acids Res., 31, e143, 2003; Xiong et al, Nucleic Acids Research, 32(12), e98, 2004) (FIG. 11). While most assembly protocols start with pools of overlapping synthesized oligos and end with PCR amplification of the assembled gene, the pathway between those two points can be quite different. In the case of LCR, the initial oligo population is required to have phosphorylated 5′ ends that allow Pfu DNA ligase to covalently connect these “building blocks” together to form the initial template. PCR assembly, however, makes use of unphosphorylated oligos, which undergo repetitive PCR cycling to extend and create a full length template. Additionally, the LCR process requires oligo concentrations in the μM (10⁻⁶) range whereas both single stranded and double stranded PCR options have concentration requirements that are much lower (nM, 10⁻⁹ range).

Published synthesis attempts have used oligos ranging in size from 20-70 bp, assembling through hybridization of overlaps (6-40 bp). Since many factors are determined by the length and composition of oligos (Tm, secondary structure, etc.), the size and heterogeneity of this population could have a large effect on the efficiency of assembly and quality of assembled genes. The percentage of correct final DNA product relies on the quality and number of “building block” oligos. Shorter oligos have lower mutated rate compared with that of longer oligos, but more oligos are required to build the DNA product. Besides, the reduced overlaps of shorter oligos results in lower Tm of the annealing reaction, which promotes non-specific annealing, and reduce the efficiency of the assembly process.

A time varying thermal field refers to the time regulated heating of the microfluidic device to allow PCR amplification or PCA reactions to occur. The time varying thermal field may be applied externally, for example by placing the microfluidic device on top of a thermal heating block, or integrated within a microfluidic device, for example as a thin film heater located immediately below the PCA and PCR chambers. A temperature controller varies the temperature of the heating element in conjunction with a temperature sensor linked to a heater element, or integrated into the reaction chamber. A timer varies the duration of heat applied to the reaction chambers. The time varying thermal field may also be applied to regulate other aspects of the microfluidic device, for example in actuating temperature responsive hydrogel valves.

The temperature of the thermal field may vary according to the denaturation, annealing and extension steps of PCR or PCA reactions. Typically, nucleic acids are denatured at about 95° C. for 2 min, followed by 30 or more cycles of denaturation at 95° C. for 30 sec, annealing at 40-60° C. for 30 sec and extension at about 72° C. for 30 sec, and a last extension of 72° C. for 10 min. The duration and temperatures used may vary depending on the composition of the oligonucleotides, PCR primers, amplified product size, template, and the reagents used, for example the polymerase.

Polymerases are enzymes that incorporate nucleoside triphosphates, or deoxynucleoside triphosphates, to extend a 3′ hydroxyl terminus of a PCR primer, an oligonucleotide or a DNA fragment. For a general discussion concerning polymerases, see Watson, J. D. et al, (1987) Molecular Biology of the Gene, 4th Ed., W. A. Benjamin, Inc., Menlo Park, Calif. Suitable polymerases include, but are not limited to, KOD polymerase; pfu polymerase; Taq-polymerase; E. coli DNA polymerase I, “Klenow” fragment, T7 polymerase, T4 polymerase, T5 polymerase and reverse transcriptase, all of which are known in the art. A polymerase having proof-reading capability such as pfu and pyrobest may be used to replicate DNA with high fidelity. Pfu DNA polymerase possesses 3′ to 5′ exonuclease proof-reading activity, thus it may correct nucleotide mis-incorporation errors.

### PCR Assembly (PCA)

PCR assembly uses polymerase-mediated chain extension in combination with at least two oligonucleotides having complementary ends which can anneal such that at least one of the polynucleotides has a free 3′-hydroxyl capable of polynucleotide chain elongation by a polymerase (e.g., a thermostable polymerase such as Taq polymerase, VENT™ polymerase (New England Biolabs), KOD (Novagen) and the like). Overlapping oligonucleotides may be mixed in a standard PCR reaction containing dNTPs, a polymerase, and buffer. The overlapping ends of the oligonucleotides, upon annealing, create regions of double-stranded nucleic acid sequences that serve as primers for the elongation by polymerase in a PCR reaction. Products of the elongation reaction serve as substrates for formation of a longer double-strand nucleic acid sequences, eventually resulting in the synthesis of full-length target sequence. The PCR conditions may be optimized to increase the yield of the target long DNA sequence.

Various PCR based methods have been described to synthesize genes from oligonucleotides. These methods are the thermodynamically balanced inside-out (TBIO) method (Gao et al, Nucleic Acids Research, 31:e143, 2003), successive PCR (Xiong et al, Nucleic Acids Research, 32:e98, 2004), dual asymmetrical PCR (DA-PCR) (Sandhu et al, Biotechniques, 12:14, 1992), overlap extension PCR (OE-PCR) (Young and Dong, Nucleic Acids Research, 32:e59, 2004; Prodromou and Pearl, Protein Eng., 5:827, 1992) and PCR-based two step DNA synthesis (PTDS) (Xiong et al, Nucleic Acids Research, 32:e98, 2004), all of which are incorporate by reference herein and can be adapted to assemble a PCR template in a microfluidic device.

DA-PCR is a one-step process for constructing synthetic genes. Four adjacent oligonucleotides 17-100 bases in length with overlaps of 15-17 bases are used as primers in a PCR reaction. The quantity of the two internal primers is highly limited, and the resultant reaction causes an asymmetric single-stranded amplification of the two halves of the total sequence due to an excess of the two flanking primers. In subsequent PCR cycles, these dual asymmetrically amplified fragments, which overlap each other, yield a double-stranded, full-length product.

TBIO synthesis requires only sense-strand primers for the amino-terminal half and only antisense-strand primers for the carboxy-terminal half of a gene sequence. In addition, the TBIO primers contained identical regions of temperature-optimized primer overlaps. The TBIO method involves complementation between the next pair of outside primers with the termini of a fully synthesized inside fragment. TBIO bidirectional elongation must be completed for a given outside primer pair before the next round of bidirectional elongation can take place.

Successive PCR is a single step PCR approach in which half the sense primers correspond to one half of the template to be assembled, and the antisense primers correspond to the second half of the template to be assembled. With this approach, bidirectional amplification with an outer primer pair will not occur until amplification using an inner primer pair is complete.

PDTS involves two 2 steps. First individual fragments of the DNA of interest are synthesized: 10-12 60 mer oligonucleotides with 20 bp overlap are mixed and a PCR reaction is carried out with pfu DNA polymerase to produce DNA fragments that are ˜500 bp in length. And second, the entire sequence of the DNA of interest is synthesized: 5-10 PCR products from the first step are combined and used as the template for a second PCR reaction with pyrobest DNA polymerase, with two outermost oligonucleotides as primers.

Although PCR assembly using oligonucleotides of 20-70 bp work well for short DNAs, there may be a limit to the maximum number of oligonucleotides that can be assembled within a single reaction. This may impose a size limit on the double stranded DNA product. A solution to this problem is to make the DNA in series. In this scheme, multiple smaller DNA segments are synthesized in parallel in separate chambers, in multiple chips, or in series and then introduced together as precursors for the PCA reaction for assembly into a “larger” DNA fragment for subsequent PCR amplification. In other words, PCR assembly using oligonucleotides would result in a template (a first-run template) for PCR amplification. A number of first-run templates so produced may serve as precursors for PCA assembly of DNA fragments larger than the first-run templates, thus producing second-run templates. In turn, the second-run templates may serve as the precursors for the assembly of a third-run template, and so on. The approach may be repeated until the desired DNA is obtained.

The oligonucleotides used in the synthesis reaction's are single stranded molecules for assembling nucleic acids that are longer than the oligonucleotide itself. An oligonucleotide may be from 10-200 bp in length but more commonly from 30-100 bp, 40-80 bp, 30-60 bp, and most commonly 20-70 bp in length. A PCA chamber containing a plurality of oligonucleotides refers to the pool of oligonucleotides necessary to produce a template corresponding to a gene or to a DNA fragment. Note that when the synthesis reactions and devices are used in series, the PCA chamber in the subsequent series of reactions would contain a pool of DNA fragments instead oligonucleotides for assembly into templates for PCR.

Oligonucleotides are usually synthesized. Oligonucleotides may be synthesized on a solid support in an array format, e.g., a microarray of single stranded DNA segments synthesized in situ on a common substrate where each oligonucleotide is synthesized on a separate feature or location on the substrate. Arrays are well known in the art; they may be constructed, custom ordered, or purchased from a commercial vendor. Methods and techniques applicable to oligonucleotide synthesis on a solid support, e.g., in an array format have been described, for example, in WO 00/58516 and Zhou et al, Nucleic Acids Res. 32: 5409-5417 (2004).

Oligonucleotides may be synthesized on one or more solid supports. Exemplary solid supports include, for example, slides, beads, chips, particles, strands, gels, sheets, tubing, spheres, containers, capillaries, pads, slices, films, plates, polymers, or a microfluidic device. Further, the solid supports may be biological, nonbiological, organic, inorganic, or combinations thereof. On supports that are substantially planar, the support may be physically separated into regions, for example, with trenches, grooves, wells, or chemical barriers (e.g., hydrophobic coatings, etc.).

Oligonucleotides may be attached to a solid support through a cleavable linkage moiety. For example, the solid support may be functionalized to provide cleavable linkers for covalent attachment to the oligonucleotides. The linker moiety may be of six or more atoms in length. Alternatively, the cleavable moiety may be within an oligonucleotide and may be introduced during in situ synthesis. A variety of cleavable moieties are available in the art of solid phase and microarray oligonucleotide synthesis (see for example, Pon, Methods Mol. Biol. 20:465-496, 1993; Verma et al, Annu. Rev. Biochem. 67:99-134, 1998; U.S. Pat. Nos. 5,739,386; 5,700,642; and 5,830,655; and U.S. Patent Publication Nos. 2003/0186226 and 2004/0106728). A suitable cleavable moiety may be selected for example, to be compatible with the nature of the protecting group of the nucleoside bases, the choice of solid support, and/or the mode of reagent delivery.

In one aspect, the oligonucleotides may be provided on a solid support for use in the microfluidic device, for example, as part of the PCA reaction chamber. Alternatively, oligonucleotides may be synthesized and subsequently introduced into a microfluidic device. Because the quantity of oligonucleotides provided by DNA microarrays is usually less, we contemplate the use of DNA microarrays in chambers of about 1 nL to 10 nL in microfluidic devices.

Oligonucleotide or DNA chip arrays are well known in the art (e.g. Affymetrix, Combimatrix). The oligo-nucleotides array chip is positioned in the microfluidic device such that the oligonucleotides immobilized on the DNA array chip (oligonucleotide spots) are accessible to the reaction fluids of the reaction chamber. The DNA array may be pressure-bonded or adhesive-bonded to the microfluidic device such that the integrity of the device is not compromised and that the oligonucleotide spots operates in alignment with the functional features of the microfluidic device. The DNA array and components of the microfluidic device may be pressure-bonded together, or a patterned adhesive such as a double-sided adhesive patterned with a laser, may be used to bond the surface of the microfluidic device to the DNA array surface.

Generally, the complete gene sequence is broken down into fixed length (N) oligonucleotides as appropriate, as discussed above. The oligonucleotide length is typically 20-70 bases. The length of the overlap between sub-sequences is commonly at N/2 but may vary from 6-40 bp, specifically 10-20 bp and 20-30 bp of overlap. The amount of partial base complementarity may vary depending on the assembly method used. For the overlapping gene assembly method demonstrated here, the PCA oligonucleotides overlap at both the 5′ and 3′ ends, except those forming the ends of the resulting PCR template. Base pair mismatches between oligonucleotides may affect hybridization depending on the nature of the mismatch. Mismatches at or near the 3′ end of the oligonucleotide may inhibit extension. However, a G/C rich region of overlap may overcome mismatches thus resulting in templates containing errors. Accordingly, consideration of the overlap sequence, melting temperature, potential for cross-hybridization and secondary structure in oligonucleotide design is necessary.

For simultaneous amplification and assembly of 10⁵ or more sequences, synthesis is unlikely to proceed uniformly (Kong et al. Nucleic Acids Res, 35(8):e61, 2007). Multiplex gene synthesis has been demonstrated from an oligonucleotide pool containing ˜600 distinct oligo-nucleotides (Tian et al., Nature, 432: 1050-1054, 2004). An oligonucleotide may be 10-200 bp in length but more commonly from 20-70 bp in length. Suitable concentrations of the PCR assembly oligonucleotides range from 5-25 nM, specifically 5-20 nM, 5-15 nM, 10-20 nM, and preferably about 10 nM. PCR assembly oligonucleotides are designed with consideration to melting temperature, potential for cross-hybridization and secondary structure in the formation of hairpins or dimers.

Template as used herein refers to a nucleic acid sequence resulting from a PCR assembly reaction and serves as the target nucleic acid for the reproduction of the complementary strand by PCR. Typically, following an assembly reaction, the PCR assembly products are double stranded DNA of variable sizes due perhaps to incomplete assembly and/or concatamers, resulting in a ladder of products as visualized by gel electrophoresis. In some embodiments, a first-run template is assembled from oligo-nucleotides. In other embodiments, a second-run template is assembled from DNA fragments comprising at least two first-run templates, the two templates being the PCR reaction products, optionally purified and error-filtered, obtained from the first two runs. A third-run template is assembled from DNA fragments comprising at least two second-run templates, and so on.

### Polymerase Chain Reaction (PCR)

PCR, as is well known in the art (e.g. U.S. Pat. Nos. 4,683,195; 4,683,202; and 4,965,188; all incorporated herein by reference) is used to increase the concentration of a target nucleic acid sequence in a sample without cloning, and requires the availability of target sequence information to design suitable forward and reverse oligonucleotide primers which are typically 10 to 30 base pairs in length. A molar excess of the primer pair is added to the sample containing the desired target or template. The two primers are complementary to 5′ and 3′ sequences of the template respectively. The mixture is first heated to denature the double stranded template and then cooled to anneal the primer to the template. Following annealing, a suitable polymerase can bind to the primer/template hybrids and extend the primers along the single stranded template, adding bases at the 3′-OH end of the primer, so as to form a complementary strand. In the presence of both forward and reverse primers, a complete copy of the original double stranded target is made. The number of cycles of denaturation, hybridization, and polymerase extension may vary as needed to amplify the template.

PCR primer refers to a sequence of a nucleic acid that is complementary to a known portion of the template sequence for use in PCR amplification. A PCR primer is a single-stranded polynucleotide or polynucleotide conjugate capable of acting as a point of initiation for template-directed DNA synthesis in the presence of a suitable polymerase and cofactors. Primers are typically from 10 to 30 nucleotides in length, or longer. The term “primer pair” refers to a set of primers including a 5′ “forward” or “upstream” primer that hybridizes with the complement of the 5′ end of the DNA template to be amplified and a 3′ “reverse” or “downstream” primer that hybridizes with the 3′ end of the sequence to be amplified.

In the present invention, PCR primers target the sequences at the 5′ and 3′ ends of the template obtained by PCA. PCA oligonucleotides are single stranded oligonucleotides used to assembly a double stranded DNA template for subsequent PCR amplification. PCR primers are single-stranded polynucleotides for amplifying the whole of the full length double stranded DNA template.

The PCR precursor mix comprises reagents at concentrations necessary for PCR amplification as well known in the art. The PCR precursor mix includes for example, dNTPs, polymerase, buffer and 0.4 μM PCR primers.

A time-varying thermal field is applied for PCR amplification and typically includes: an initial denaturation at about 95° C. for 2 min, followed by 30 or more cycles of denaturation at 95° C. for 30 sec, annealing at 40-60° C. for 30 sec and extension at about 72° C. for 30 sec, and a last extension of 72° C. for 10 min. The time and temperature may vary depending on the nature of the template, the primer, polymerase or other reagents used.

### Purification

PCR products may be purified in ways adaptable to a microfluidic device.

Solid phase extraction is an important and widely used sample preparation technique, which allows the purification, pre-concentration of samples, and/or buffer exchange (Tan et al, Anal. Chem., 75:5504-5511, 2003). The purification of nucleic acids can be done with DNA-binding polymers such as polyethylene glycol. Solid-phase extraction on silica resins (Wolfe et al, Electrophoresis, 23:727-733, 2002) is a common technique. Extraction is achieved because nucleic acids have the tendency to bind to silica in the presence of a high concentration of chaotropic salt (Boom et al, J. Clin. Microbiol., 28:495-503, 1990). The extracted nucleic acids are subsequently eluted in an aqueous low-salt buffer and concentrated into a very small volume.

Solid phase purification may utilize magnetic beads coated with silica, sol-gel silica, silica particles, or microfabricated silicon structure. Note that methods using silica, sol-gel silica, silica particles, or microfabricated silicon structure would involve an additional process to immobilize the silica material within the chamber to withstand fluidic flow. Accordingly magnetic beads are preferred because an external magnet can be applied to capture and agitate the beads. The magnetic beads also provide better DNA extraction efficiency and DNA loading capability.

Certain embodiments of the present invention includes the use of magnetic beads coated with silica. The beads are introduced into the device via an inlet. The silica coated magnetic beads are manipulated in the device with a magnet and reversibly engage PCR products. The PCR products may be eluted from the silica coated magnetic beads using an aqueous low-salt buffer. The low-salt elution buffer may be different from that used in the PCR reaction.

Magnetic bead refers to a nanoparticle, bead, or microsphere, or by other terms as known in the art, having at least one dimension, such as apparent diameter or circumference, in the micron or nanometer range. An upper range of such dimensions is 600 μm, but typically apparent diameter is under 200 μm, and may be 1-50 μm or 5-20 nm, while not limited to such. Such particles may be composed of, contain cores of, or contain granular domains of, a paramagnetic or superparamagnetic material, such as the Fe2Cb and Fe304 (α-Fe crystal type), α′-FeCo, ε-Cobalt, CoPt, CrPt3, SmCos, Nickel and nickel alloys, Cu2MnAI, α-FeZr, Nd2Fe)4B, NoTi, for example. These materials may be formed into particles, beads or microspheres with binders such as polymers, silica, or other known materials.

Solid phase extraction methods for DNA extraction are successfully miniaturized and incorporated in micro-fluidic chips. The sol-gel/silica bead mixtures in particular have very good extraction efficiencies and reproducibility in microfluidic systems (Wolfe et al, Electrophoresis, 23:727-733, 2002).

### Error Filtering

Current oligonucleotide synthesis technologies produce by-products that are either prematurely terminated, or more detrimentally, contain internal deletions in the sequence that introduce errors to the final DNA (Carr et al, Nucleic Acids Research, 32(20), e162, 2004; Hoover and Lubkowski, 30(10), Nucleic Acids Research, pp. e43, 2002). Although synthetic oligonucleotides can be purified first using polyacrylamide gel electrophoresis (PAGE) or high-performance liquid chromatography (HPLC) methods, these processes will dramatically increase the cost and time. To effectively reduce the error rate, an error-filtering or error-correction process such as those based on enzymatic affinity capture and selectively mismatch cleavage may be used.

Known techniques for DNA error correction may be incorporated into a microfluidic device for use before or after PCR amplification. Exemplary techniques for the removal of mismatched bases include but are not limited to enzymatic affinity capture, enzymatic mismatch cleavage, consensus shuffling and oligonucleotide hybridization.

In consensus shuffling, DNA is fragmented and mismatched fragments are removed upon binding to an immobilized mismatch binding protein (e.g. MutS). PCR assembly of the remaining fragments yields a new population of full-length sequences enriched for the consensus sequence of the input population.

In oligonucleotide hybridization, oligonucleotides are annealed to immobilized oligonucleotides that are complementary to the PCR assembly oligonucleotides under annealing conditions. Once hybridized, the oligonucleotides that are not hybridized, and thus likely to contain mutations, are wash away.

In enzymatic affinity capture, mutated DNA are captured and removed from the product solution using DNA mismatch-binding proteins such as MutS and T4E7. Enzymes such as T4E7, endonuclease V and T7E1 can recognize and cut the DNA at mismatch site. Carr et al, Nucleic Acids Research, 32(20), e162, 2004 showed that MutS can reduce error by >15-fold relative to conventional gene synthesis techniques, yield DNA with one error per 10 k base pairs. The MutS-bound mutated DNA segments are separated from the correct product using gel electrophoresis. To incorporate this affinity capture method into microfluidic structure, we describe beads-based solid-phase chromatography with immobilized histidine-tagged MutS (Bi et al, Anal. Chem., 75, 4113-9, 2003). We contemplate using T4E7 (Taylor and Deeble, Genetic Analysis: Biomolecular Engineering, 14, 181-6, 1999) for error filtration which has mismatch binding preference complementary to MutS. Certain embodiments of the present invention include on-chip error filtration employing any of the error filtering methods disclosed herein or combinations thereof.

In selectively mismatch cleavage, mutated DNA are captured and cleaved into smaller segments using the endonuclease proteins such as the combination of MutH, MutS & MutL (Smith and Modrich, Proc. Natl. Acad. Sci., 94(13), 6847-50, 1997), and the T7E1 (Youil et al, Proc. Natl. Acad. Sci., 92, 87-91, 1995). The cleaved segments are then filtrated from the product based on the segment size by micromachined electrophoresis. This process can be incorporated into microfluidic structure without immobilizing the protein on solid support.

MutS refers to a DNA-mismatch binding protein that recognizes and binds to a variety of mispaired bases and small (1-5 bases) single-stranded loops. Exemplary MutS proteins include, but are not limited to, polypeptides encoded by nucleic acids having the following GenBank accession Nos: AF146227 (Mus musculus), AF193018 (Arabidopsis thaliana), AF144608 (Vibrio parahaemolyticus), AF034759 (Homo sapiens), AF104243 (Homo sapiens), AF007553 (Thermus aquaticus caldophilus), AF109905 (Mus musculus), AF070079 (Homo sapiens), AF070071 (Homo sapiens), AH006902 (Homo sapiens), AF048991 (Homo sapiens), AF048986 (Homo sapiens), U33117 (Thermus aquaticus), U16152 (Yersinia enterocolitica), AF000945 (Vibrio cholarae), U698873 (Escherichia coli), AF003252 (Haemophilus influenzae strain b (Eagan), AF003005 (Arabidopsis thaliana), AF002706 (Arabidopsis thaliana), L10319 (Mouse), D63810 (Thermus thermophilus), U27343 (Bacillus subtilis), U71155 (Thermotoga maritima), U71154 (Aquifex pyrophilus), U16303 (Salmonella typhimurium), U21011 (Mus musculus), M84170 (S. cerevisiae), M84169 (S. cerevisiae), M18965 (S. typhimurium) and M63007 (Azotobacter vinelandii). Exemplary mutS homologs include, for example, eukaryotic MSH2, MSH3, MSH4, MSH5, and MSH6 proteins (see U.S. Pat. Nos. 5,858,754 and 6,333,153). The term is meant to encompass prokaryotic MutS proteins as well as homologs, orthologs, paralogs, variants, or fragments thereof. The term also encompasses homo- and hetero-dimers and multimers of various MutS proteins.

### Compact Disk (CD)—Based Gene Assembly

Gene assembly as a whole involves several serial steps such as synthesizing oligonucleotides, purifying oligos, assembling the oligo segments by ligation and/or polymerase cycling, removing the incorrect sequences (filter out the errors), and amplifying the gene by polymerase chain reaction. It is desirable to integrate these gene assembly processes and required components into a single devise such as a compact disk format (referred to as Gene-CD) to provide an automatic gene synthesizer.

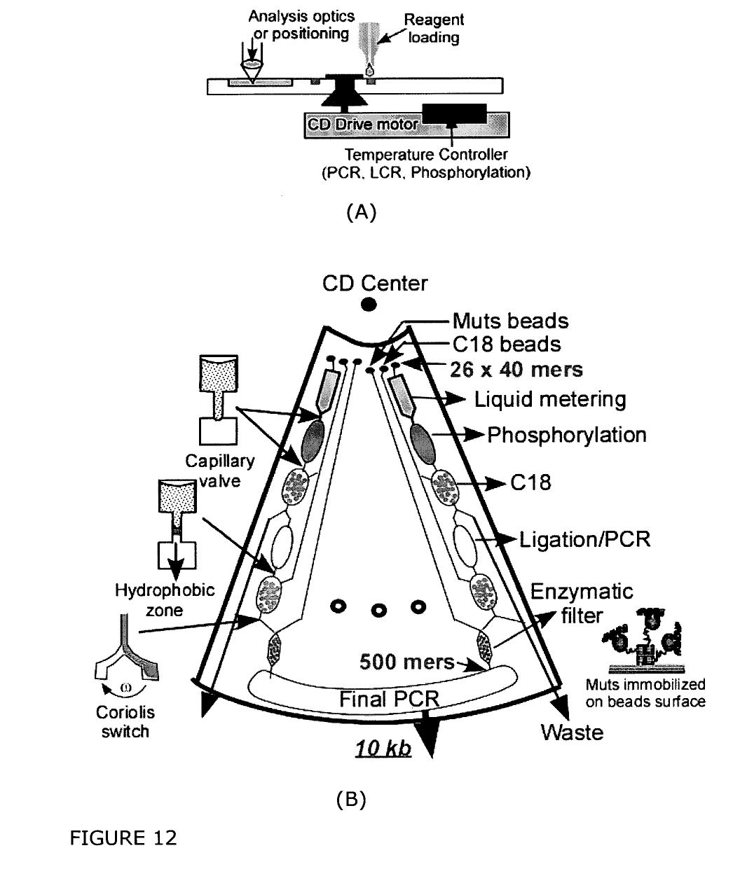

One device format is a compact disk (CD)-based microfluidic (FIG. 12). This approach provides several advantages on fluidic delivery and fluidic regulation. The fluidic packets can be pre-metered, and moved from chamber to chamber by using centrifuge force which eliminates the external pumps (Zoval and Madou, Proc. of the IEEE, 92(1), 140-53, 2004). The fluidic flow rate can be controlled easy with programmable rotational speed. Also, capillary valve made by a sudden expansion in channel diameter, and hydrophobic valve made by the application of hydrophobic material to a zone in the channel can be incorporated with the rest of the structures for precise fluid control. Moreover, the fluidic in a Y-structure can be switched between two channel outlets by using Coiolis force selected by the direction of rotation and speed (Brenner et al, Lab on a Chip, 5, 146-50, 2005). The reagents exchange among phosphorylation, ligase chain reaction (LCR), polymerase chain reaction (PCR), and error filtering can be solved using reverse-phase or ion-exchange chromatography with column filled with C18 and silica beads respectively and carefully controlled flow rate (Jemere et al, Electrophoresis, 23, 3537-44, 2002). Enzymatic error filter to remove DNA with incorrect sequence can be incorporated into fluidic structure with column filled with MutS and T4E7 immobilized beads. These beads are introduced to the columns and localized by using a simple restriction region after the microfluidic device is fabricated. Finally, the temperature control for performing phosphorylation, LCR, and PCR will be achieved using external heater or infrared as a heating source (Giordano et al, Analytical Biochemistry, 291, 124-32, 2001).

For performing large gene (e.g. 10 kb) assembly, multiple microfluidic columns can be incorporated into one Gene-CD and operated simultaneously to achieve maximum efficiency. Long DNA sequence will be divided into segments with each segment assembled at each separate fluidic column, and then linked together at the final PCR step.

For rapid protyping, fluidic components may be fabricated using soft lithography or CNC milling on polycarbonate material (Lee et al, Biomedical Microdevices, 3(4), 339-351, 2001).

### (VII) EXEMPLIFIED EMBODIMENTS

### Microfluidic Device Fabrication

Instead of using SU-8-based lithography process to create the polydimethylsiloxane (PDMS) casting mold, we have adopted a three-dimensional (3D) rapid prototype method that printed 3D structure using photosensitive resin. The 3D structure was designed in SolidWorks and transferred to the Eden 350 (Objet Geometries), which printed photopolymer material (FullCure 720) and support material (FullCure 705) layer by layer. The photopolymer layer was cured by UV light immediately after it was printed. Upon completion, the fabricated structure was soaked in 25% tetramethylammonium hydroxide (TMAH) solution for 3 h to remove the support material designed for supporting the printed geometries. The microfluidic mold was soaked in water for 1 h to wash away TMAH. This method provided a resolution of 42 μm in the x-axis and y-axis, and a resolution of 16 μm in the z axis, well-suited for generating thick and multilevel structures without lithographic process. Conversely, other rapid prototype methods such as liquid phase photopolymerization (Khoury et al, Lab Chip, 2002, 2, 50) and contact liquid photolithographic polymerization (Hutchison et al, Lab Chip, 2004, 4, 658) utilized photomasks to facilitate construction of structures with superior resoultion. Two-level mold was designed with different heights for connection channels (height: 0.2 mm; width: 0.2 mm) and chambers (height: 0.5 mm) to minimize the dead volume of connection channels.

Poly(dimethylsiloxane) (PDMS) precursor was prepared by mixing Sylgard 184 base and Sylgard 184 curing agent in a 10:1 volume ratio. The precursor was poured into the mold, degassed in vacuum chamber for 30 min, and cured in a convection oven at 75° C. for 3 h. The 3-mm thick PDMS slab was then peeled off from the mold, and connection holes were pierced. The microfluidic device was assembled by bonding PDMS and silicon substrate (500 μm-thick). Both the PDMS and silicon substrate were treated with electrical discharges treatment (Kim et al, J. Colloid Interface Sci., 2001, 244, 200). Finally, the device was cured in an oven at 75° C. for 2 h to ensure irreversible bonding between PDMS and the silicon substrate.

To prevent sample evaporation, the bonded device was deposited with a 2 μm-thick Parylene C using the PDS 2010 Parylene Deposition System (SCS, USA). The vapor deposited Parylene C created a barrier to control the water vapor diffusion. Parylene also passivated the inner surface of the device in preventing unwanted protein absorption (Shin et al, J. Micromech. Microeng., 2003, 13, 768; Shih et al Actuators A, 2006, 126, 270).

### Preparation of Hydrogel Valves

Thermosensitive hydrogel valves were selected for fluidic regulation and confining PCR reaction. Hydrogel was synthesized following the method suggested by van der Linden et al, Lab Chip, 2004, 4, 619. Temperature-sensitive monomer N-isopropylacrylamide (NIPAAM, 286 mg), N,N′-methylene bisacrylamide (BIS, 7.88 mg) crosslinker and 2,2′-dimethoxy-2-phenyl acetophenone (DMPAP, 18.86 mg) photoinitiator were mixed in 500 μL of dimethylsulfoxide (DMSO), generating a precursor solution containing 2% BIS crosslinker. The precursor was purged with nitrogen to remove oxygen, and wrapped with aluminum foil to avoid unwanted photo-polymerization. All the chemicals were purchased from Sigma-Aldrich (Singapore).

The mixture was then injected into the fabricated chip using a 1-ml syringe through the access holes, and photopolymerized in situ at 32° C. with a chromium mask defining the exposed area. The sample was then irradiated at a wavelength of 365 nm (dose: 252 mJ/cm²) using OmniCure Series 2000 UV illumination system (EXFO, Canada). After ultraviolet exposure, the device was placed on a hotplate at 60° C. to keep hydrogel valves open, and the unpolymerized precursor was removed with de-ionized water at a flow rate of 500 μL/min for 40 min using a syringe pump (74900 Series, Cole-Parmer Instrument Company). Finally, the device was baked at 75° C. for 3 h in an oven to dry its inner surface and hydrogel valves.

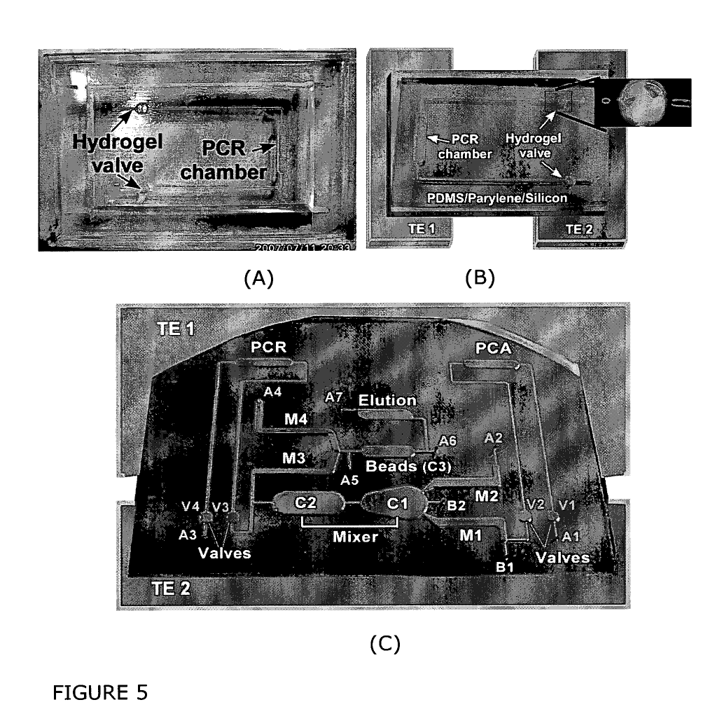

The NIPAAm-based hydrogel is thermosensitive with a lower critical solution temperature (LCST) of 32° C. (van der Linden et al, Lab Chip, 2004, 4, 619). The hydrogel would swell at temperatures below 32° C., blocking the fluidic channel. At a temperature above 32° C., the polymer chains became hydrophobic, causing the hydrogel to shrink and allowing fluid flowing through. The opening and closing of valves were controlled by varying the temperature between 4° C. and 60° C. An example of the integrated hydrogel valves in the microfluidic device fabricated with the printed photosensitive resin mold is shown in FIG. 5.

### PCR Thermal Cycling

The PCR was performed by using a home-made thermal cycler, which included a fan, a thermoelectric (TE) heater/cooler (9501/127/030, FerroTec) and a thermoelectric control kit (FerroTec, USA) consisting of FTA600 H-bridge amplifier, FTC100 temperature controller and FTC control software. The thermoelectric heater was powered by the FTA600 amplifier, which was controlled by the FTC100 temperature controller. A T-type thermocouple (5TC-TT-T-40-36, OMEGA Engineering) was mounted on the TE heater to measure the temperature, and used as a feedback to the FTC100 temperature controller. The temperature difference between the thermoelectric heater and actual temperature inside the PCR chamber was calibrated using a calibration chip, which has identical dimensions as the actual device but a thermocouple embedded inside the PCR chamber filled with PCR mixture. The temperature drop between the heater surface and inside the chamber was noted in the FTC control software and compensated during the operation. The desired temperature profile was programmed into a computer through the FTC control software, which controlled the FTC100 temperature controller using a PID (proportional-integrative-derivative) algorithm to optimize the temperature response time.

### Gene Assembly and Amplification

Published gene segment of GFPuv with a total length of 760 bp (sequence 261-1020 with T357C, T811A and C812G base substitutions) was selected for synthesis. It was assembled using 37 of 40-mer and 2 of 20-mer oligo-nucleotides with 20 bp overlap (Table 2; Binkowski et al, Nucleic Acids Res., 2005, 33, e55). The PCR synthesis reactions were performed both within the microfluidic devices and in the standard 0.2-ml PCR tubes with a commercial thermal cycler (DNA Engine PTC-200, Bio-Rad) for comparison of the synthesis performance. Synthesis via PCR was performed either as a one-step process, combining assembly PCR and amplification PCR into a single stage, or as a two-step process with separate stages for assembly and amplification. The one-step process in PCR tube was conducted with 50 μl of reaction mixture including 1×PCR buffer (Novagen), 1 mM of MgSO4, 0.25 μM each of dNTP (Stratagene), 5-25 nM of oligonucleotides, 0.1-0.4 μM of forward and reverse primers, and 1 U KOD Hot Start (Novagen). The PCR was performed under the following conditions: 2 min of initial denaturation at 95° C.; 30 cycles of 95° C. for 30 sec, annealing at 50° C. for 30 s, 72° C. for 30 sec, and last extension at 72° C. for 10 min. The PCR protocol of the two-step process was essentially the same as that for the one-step process. For PCR assembly, 5-25 nM of oligonucleotides were used without the forward and reversed primers. For gene amplification, the assembled product was 2× diluted with fresh amplification reaction mixture containing a final primers concentration of 0.4 μM. Microfluidic syntheses were conducted with the same PCR conditions in adjusted chamber volume. All processes were performed with desalted oligonucleotides from Research Biolabs (Singapore) without additional purification.

### Solid-Phase Buffer Exchange

Solid-phase buffer exchange was conducted using the magnetic beads based PCR purification method (ChargeSwitch PCR clean-up kit, Invitrogen) on microfluidic devices, and in standard 0.2-mL PCR tubes (as control) with the synthesized PCR product and 100 bp DNA ladder (New England, 170 ng/μL) as control.

For the control experiment performed in PCR tube, the 100 bp DNA ladder or PCR-synthesized product (7 μL) was mixed with 5 μL of beads and 11 μL of purification buffer (Invitrogen), and incubated for 1 min. The beads were then captured by a magnet to remove the supernatant. We washed the beads with 150 μL of washing buffer (Invitrogen) three times, and loaded 7 μL of elution buffer (10 mM of Tris-HCl, pH 8.5) to the washed beads. The elution buffer and beads were incubated at different conditions (25-80° C. for 2-3 min) to optimize the elution efficiency of bound DNA. The concentrations of the original and eluted DNA samples were measured and compared by UV-Vis spectrophotometer (ND-100, Nanoprop Technologies).

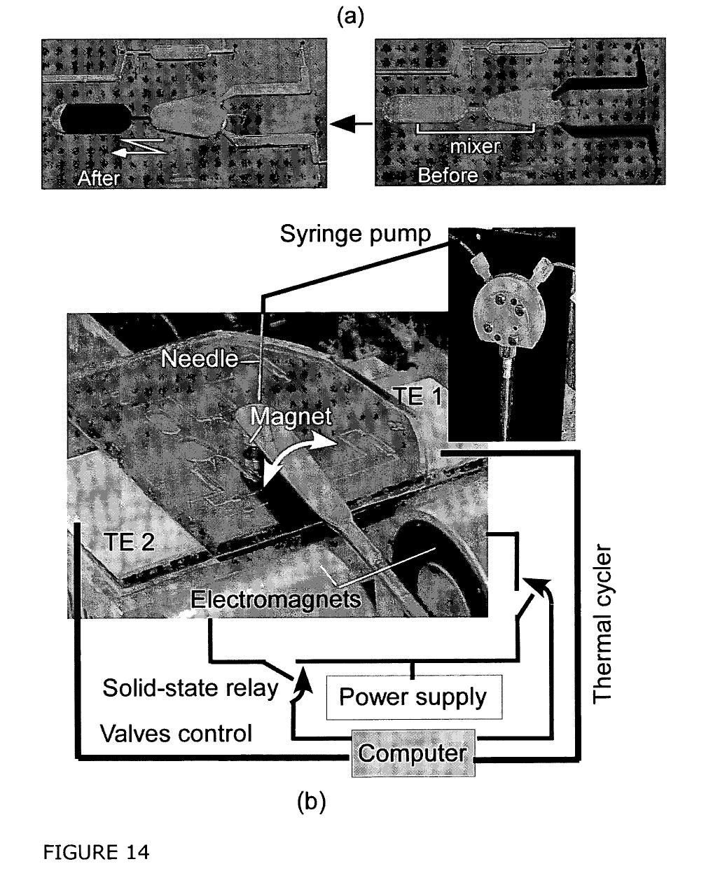

Similar process was conducted on the two-step microfluidic device (FIG. 5C). The 100 bp ladder or PCR product (7 μL) and magnetic beads (5 μL) in purification buffer (11 μL) were first loaded into M3 and M4, respectively, with a volume defined by the meter chambers. These two solutions were mixed using an external syringe pump (Cavro XLP 6000), pushed to the bead chamber (C3), and incubated for 1 min. The impurities in the bead chamber were then washed with washing buffer introduced from A5 at a flow rate of 200 μL/min for 15 min with beads captured by a permanent magnet (M1219-5, Assemtech). After washing, elution buffer (10 mM of Tris-HCl, pH 8.5, 5 μL) was introduced into the bead chamber, and incubated with the beads at 25-80° C. for 2-3 min to release the bound DNA. The magnetic beads were actively mixed at a rate of 0.5 Hz before elution using external electromagnets with the setup shown in FIG. 14b. A permanent magnet was mounted on a flexible and suspended metal arm located between two electromagnets (GMHX, Magnet-Schultz Ltd). Alternate magnetic forces were applied to the metal arm when 180° out-of-phase voltages were supplied to the electromagnets, which swung the metal arm and the mounted magnet. The electromagnets were powered through the solid-state relays (ODCM-5, Tyco) and a DC power supply (HY3003, Digimess). Electromagnetic forces were regulated through the relays using an analog voltage output board (PCI-6713, National Instruments), and a computer with a LabVIEW program (National Instruments).

### Agarose Gel Electrophoresis

Synthesized products were analyzed by 1.5% agarose gel (NuSieve® GTG®, Cambrex Corporation), stained with ethidium bromide (Bio-Rad Laboratories), and visualized using Typhoon 9410 variable imager (Amersham Biosciences). Gel electro-phoreses were performed at 100 V for 45 min with 100 bp ladder ((New England) and 5 μL of DNA samples collected from commercial thermal cycler and devices.

### DNA Sequencing

One-step and two-step overlapping synthesis products were sequenced to check the error rate. GFPuv gene synthesis products (without further PCR purification) were cloned into vector pCR®2.1-TOPO® (Invitrogen) and transformed into chemically competent TOP10 cells. After overnight growth on 1× Luria-Bertani (LB) agar plate (with 100 μg/ml of ampicillin), individual colonies were picked and grown in 1×LB media (with 100 μg/ml of ampicillin). The plasmid DNA was extracted by using QIAprep Spin Miniprep Kit (QIAGEN), and sequenced by Research Biolabs (Singapore). In total, 150 individual samples were sequenced using M13 forward and reverse sequencing primers (for one-step process: 96 from microfluidic device and 48 from 0.2-ml PCR tube; For two-step process: 54 from microfluidic device and 48 from 0.2-ml PCR tube). All sequence results were analyzed using sequence analysis tool Vector NIT, and the errors were verified by visual confirmation of the electrophoregrams of ABI PRISM® 3100-Avant Genetic Analyzer.

### Device Operation

A precision syringe pump with multi-position valve (Cavro XLP6000, Tecan Systems) was used to manipulate reagents inside the microfluidic device. This syringe pump is capable of withdrawing and dispensing reagents with a volume resolution of better than 10 mL, as controlled by a LabVIEW program (National Instruments). To control the hydrogel valves and thermal cycling simultaneously and separately, two TE modules with individual temperature controllers were used (FIG. 5). One TE module (TE 1) was located under the PCR chambers to perform the temperature cycling, and the other TE module (TE 2) was located under the hydrogel valves to control their action.

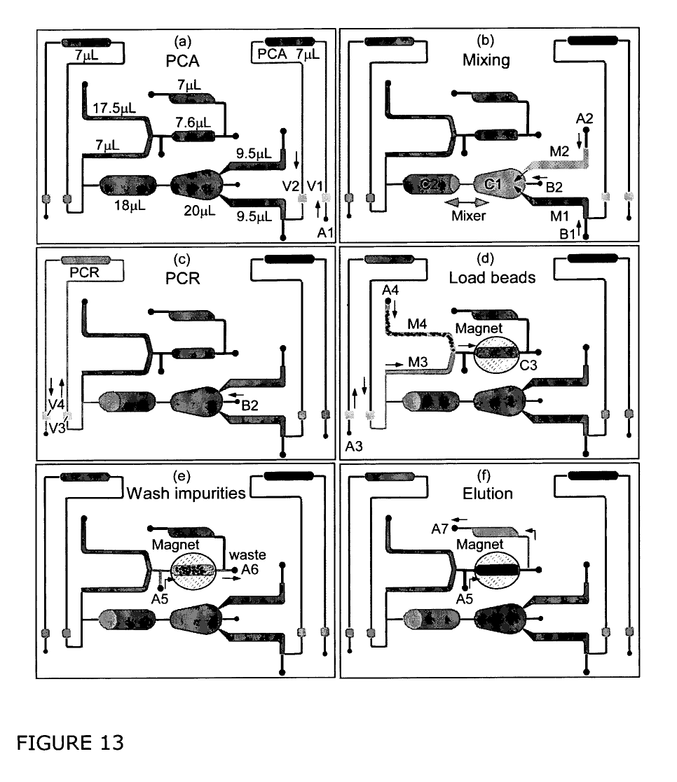

The overall device operation of the gene synthesis device was illustrated in FIG. 13 with the volume defined by each chamber. Oligonucleotide and PCR mixture was first loaded into the PCA chamber through the inlet port (A1). The solution was then sealed by the hydrogel valves (V1 and V2) and thermally cycled with the thermoelectric heater to assemble oligonucleotides. After PCA, the hydrogel valves (V1 and V2) were opened, and the solution was pumped into meter chamber M1, and simultaneously mixed with an equal volume of fresh PCR mixture containing outer primers from meter chamber M2. To enhance mixing, this mixture was shuttled between two mixing chambers (C1 and C2) five times (flow rate=120 μL/min) with the precision syringe pump at inlet port B2, and then moved to PCR chamber with hydrogel valves (V3 and V4) kept open. After PCR amplification, the hydrogel valves (V3 and V4) was open again, and the solution was moved to meter chamber M3 (through inlet port A3), and simultaneously mixed with the magnetic beads solution defined by meter chamber M4 in the beads chamber (C3). With the DNA-absorbed magnetic beads captured by a permanent magnet, the impurities solution was washed out. Finally, the elution buffer was loaded and mixed with the magnetic beads; the DNA was then released into the elution buffer. To control the flow direction, unused inlets and outlets were plugged with metal pins. For example, to direct PCA mixture to PCR chamber, the inlets (A4-A7) for solid-phase PCR purification were plugged.

Two micromixers were developed to effectively mix the PCA product with fresh PCR mixture for PCR amplification, and mix the magnetic beads with DNA solution and elution buffer for solid-phase PCR purification. The gene synthesis chip was to be developed as a bench-top instrument to perform automatic gene synthesis. To control the cost and simplify the fabrication process of these disposable chips, mixing approaches utilizing simple fluidic structures and methods were desired. FIG. 14 shows our approaches using shuttle mixing and electromagnetic mixing. In shuttle mixing, solution was shuttled between two chambers connected by a narrow channel. This narrow channel reduced the diffusion distance of two mixing reagents, and the abrupt opening at channel-chamber junctions created chaotic advection at the junctions and recirculated the flow (Gan et al, Appl. Phys. Lett., 2006, 88, 224103). Both of these features were reported to enhance mixing (Nguyen and Wu, J. Micromech. Microeng., 2005, 15, R1): FIG. 14a demonstrated the performance of the shuttling micromixer. Two colored food dyes (blue and red) were well mixed after shuttled three times between two chambers at a flow rate of 150 μL/min, pumped by a precision syringe pump. This method was effective with compact and simple fluidic structures as compared to other reported methods (Nguyen and Wu, J. Micromech. Microeng., 2005, 15, R1). Mixing was completed within 1 min in our application with a fluid volume of 19 μL. No visible air bubbles were trapped inside the solution.

Permanent neodymium rare earth magnet was utilized to capture magnetic beads in the microfluidic device (Liu et al, Anal. Chem., 2004, 76, 1824; Herrmann et al, Lab Chip, 2006, 6, 555; Grumann et al, Lab Chip, 2005, 5, 560), as it provided a strong magnetic force. However, this strong magnet could also cause the aggregation of beads (Rida and Gijs, Anal. Chem., 2004, 76, 6239), and hinder the beads from full contact with the desired biomolecules in solution. To make sure that the beads were well mixed with solution, we have developed an approach to agitate the solution inside the chamber (FIG. 14b). A permanent magnet was mounted on a flexible metal arm that was sandwiched by two electro-magnets. When out-of-phase voltages were applied to the electromagnets, alternating magnetic forces were generated, which swung the metal arm and the permanent magnet simultaneously. The swinging magnet dragged the magnetic beads and agitated the solution. This simple approach was employed to mix the elution buffer with DNA-bound magnetic beads in the final step of PCR purification at a mixing rate of 0.5 Hz.

### In Situ Hydrogel Valve

During the PCR process, the air solubility variation from 4° C. to 94° C. could create a pressure of ˜3.1 psi (Chiou et al, Anal. Chem., 2001, 73, 2018). Potential trapped air bubbles would contribute an additional pressure of 3.7 psi at 94° C. (Liu et al, Anal. Chem., 2002, 74, 3063).

Therefore, the preferred microvalve would be able to withstand at least 6.8 psi to ensure successful sealing of the PCR mixture within the chamber.

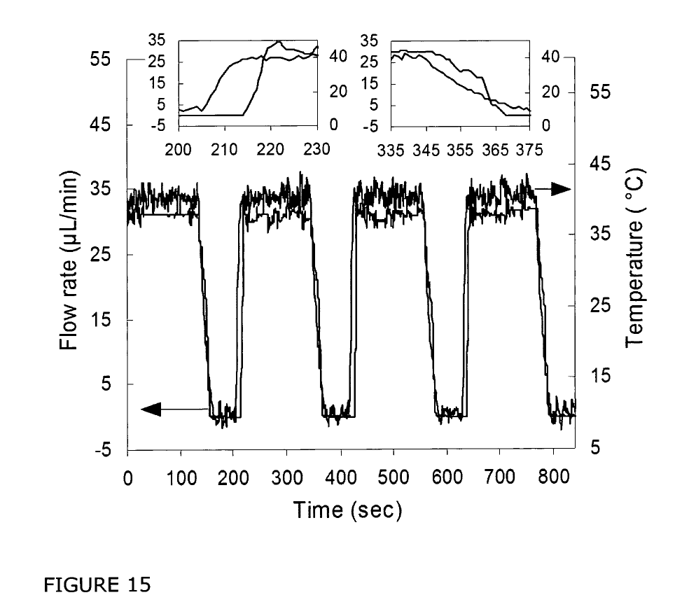

The hydrogel valves were tested prior to use on the single-chamber device (FIG. 5b) with a liquid flow meter (SLG1430, Sensirion) connected between a constant pressurized water reservoir (8 psi) and the device. The flow rate variation was monitored as the valve was subjected to repetitive cooling and heating by a thermoelectric heater underneath the device. FIG. 15 shows the valve's temperature and the flow rate as functions of time. The valve dimensions were 1.5 mm×1.5 mm×0.5 mm. At a temperature below the hydrogel's LOST (32° C.), the thermally responsive hydrogel swelled and blocked the valve, indicated by the decrease in flow rate. When the temperature was above the hydrogel's LCST, the hydrogel shrunk in volume and allowed for fluid flow through the channel. As indicated in FIG. 15, the valve functions were highly repeatable with valve's opening and closing times of ˜5 sec and ˜20 sec respectively (see inset in FIG. 15), limited by the ramping rate of the underneath heater, and the water diffusion rate of the hydrogel swelling/de-swelling process (Richter et al, Sens. Actuators B, 2004, 99, 451). The closed valve exhibited no leakage (zero flow rate) at 8 psi, showing that it was strong enough to seal the PCR chamber. Yu et al, Anal. Chem., 2003, 75, 1958 reported that in situ photopolymerized NIPAAm-based valve could withstand a pressure of up to 200 psi. Wang et al, Lab Chip, 2006, 6, 46 and Wang et al, Biomed. Microdev., 2005, 7, 313 also described the successful integration of chemically polymerized NIPAAm hydrogel valve with PCR by manual insertion of pre-synthesized hydrogel in the flow paths.

### PCR Thermal Cycling

The gene synthesis process was integrated into a chip composed of a PDMS fluidic structure on a silicon substrate. Although PDMS has a number of interesting material properties that make it superior for constructing highly integrated biological microsystems, its non-specific protein adsorption (Zhang and Xing, Nucleic Acids Res., 2007, 35, 4223; Huang et al, Lab Chip, 2005, 5, 1005) and permeability to water vapor (Prakash et al, Sens. Actuators B, 2006, 113, 398) could pose problems in performing PCR in microfluidic environment, which has a small volume and a high surface-to-volume ratio. To address these problems, we have coated the fabricated devices with 2 μm-thick parylene, which created a barrier to against water vapor diffusion and improved the surface compatibility with PCR mixture (Shin et al, J. Micromech. Microeng., 2003, 13, 768).

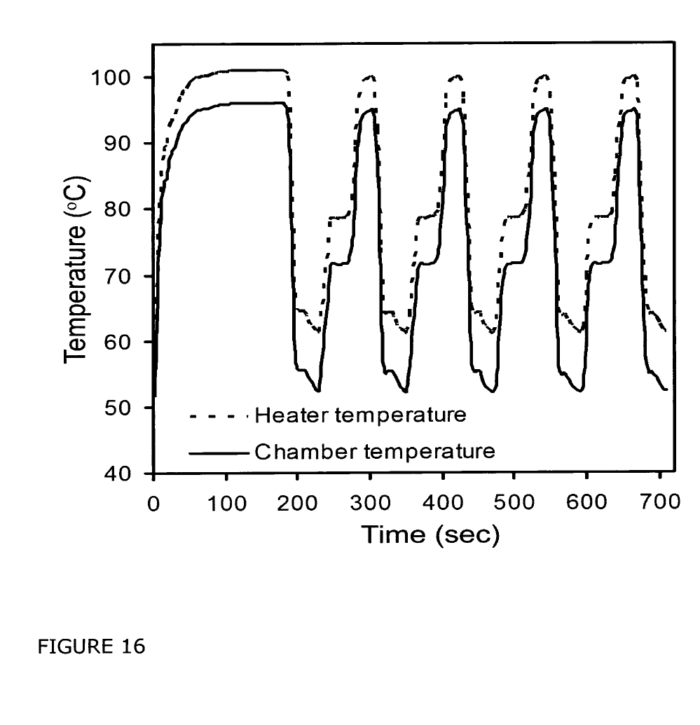

A thermoelectric module with heat sinks and fan was utilized for thermal cycling. FIG. 16 showed the temperature profiles of the thermal cycler obtained from a calibration chip, which has identical dimensions as the actual device, but has a thermocouple embedded within the PCR chamber. Temperatures at the heater surface and within the PCR chamber were measured. The temperature difference between these two locations indicated that the 500 μm-thick silicon substrate could cause a temperature drop of >5° C., which was compensated during the operation of thermal cycling. The heating and cooling rates estimated from FIG. 16 were 2.4° C./sec and 4.3° C./sec, respectively, which were faster than those in commercial thermal cycler (DNA Engine PTC-200).

The PCR chamber was designed with a volume of 7 μL. Gene synthesized by PCR methods contained both full-length DNA and intermediaries with shorter lengths. After synthesis, gel electrophoresis was usually conducted to confirm the success of the synthesis, and to separate the full-length product, which was then extracted from the gel by using gel extraction kits. Some DNA could be lost due to these steps and the pipetting process. The PCR mixture was introduced into the PCR chamber through the hydrogel valves that were kept opened by thermoelectric heater at 60° C. Once the PCR chamber was filled with the solution, the hydrogel valves was cooled to 4° C., sealing the chamber. Since silicon with high thermal conductivity was used as the device substrate, the PCR chamber and hydrogel valves were positioned apart to minimize thermal interference between the PCR thermal cycling and the valves' operation. The hydrogel valve has to be kept below the transition temperature to seal the PCR chamber during thermal cycling, which could reach a temperature as high as 95° C. One way to suppress the thermal interference and reduce the dead volume between the PCR chamber and valves was to use a polymer substrate (such as polycarbonate) (Zou et al, Sens. Actuators A, 2002, 102, 114) or an isolation trench to suppress the lateral heat flow along the substrate, as reported by Wang et al, Lab Chip, 2006, 6, 46 and Yang et al, J. Micromech. Microeng., 2005, 15, 221.

### Comparison of One-Step and Two-Step Gene Syntheses

The thermal cycler's requirement for PCR assembly was the same as the standard PCR amplification. However, the number of oligonucleotides involved in PCR assembly was much larger than in the standard PCR amplification. Full-length DNA was constructed from a pool of solution containing tens of oligonucleotides with various melting temperatures. The efficiency of successful gene synthesis relied on several important factors including the polymerase, concentrations of assembly oligonucleotides and amplification primers, and structure and properties of oligonucleotides (Cox et al, Protein Sci., 2007, 16, 379; Wu et al, J. Biotech., 2006, 124, 496).

To identify the baseline of oligonucleotide and primer concentrations, a segment of GFPuv (760 bp) was synthesized from a pool of short oligonucleotids (40 bases) using two-step PCR process by varying oligonucleotide concentration from 5 to 25 nM, and primer concentration from 0.1 to 0.4 μM; this was conducted on the commercial thermal cycler. Desired full-length product was first assembled from oligonucleotides without outer primers (PCA assembly), and then amplified by adding these primers at the second PCR (PCR amplification). To match the microfluidic device design (FIG. 5c), the PCR amplification was performed with the PCA product diluted with an equal volume of fresh amplification reaction mixture.

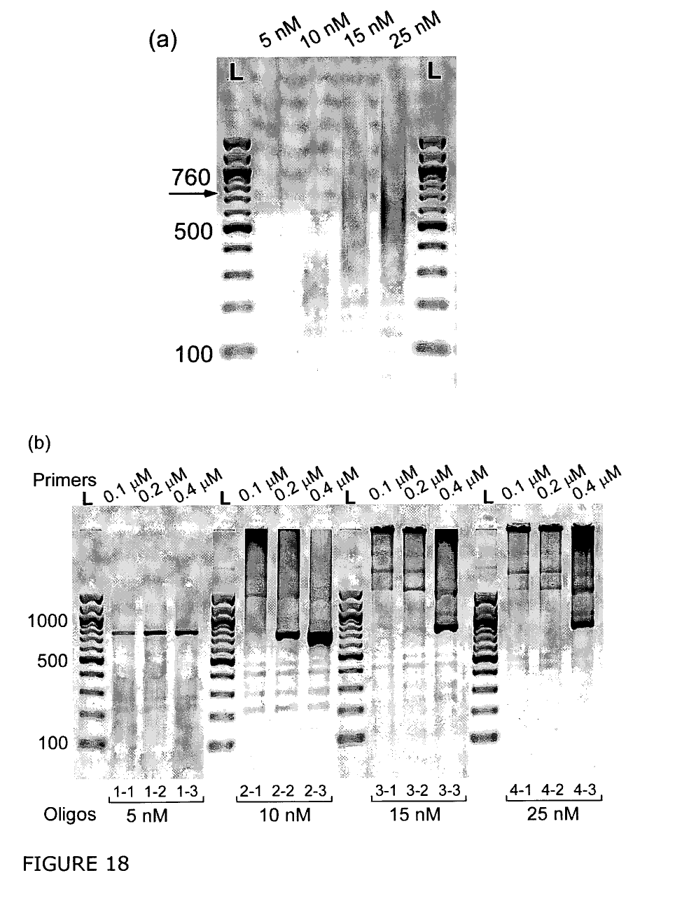

Gel electrophoresis results for PCA assembly (FIG. 18a) and PCR amplification (FIG. 18b) were illustrated for the indicated oligonucleotide and primer concentrations. The PCA has smearing gel results, indicating that the assembled product contained a spectrum of DNAs, the majority of which possessed lower molecular weights than the desired target (760 bp). For products assembled from oligonucleotide concentrations of <10 nM, the quantity of full-length DNA (760 bp) was very low and invisible in the PCA gel images, but this was effectively boosted with PCR amplification. PCR gel images showed samples 1-1 to 1-3 synthesized with an oligonucleotide concentration of 5 nM and a primer concentration of 0.1 μM, 0.2 μM and 0.4 μM, respectively. Other samples were the same as samples 1-1 to 1-3, except that the oligonucleotide concentrations used were 10 nM, 15 nM and 25 nM. Syntheses with an oligonucleotide concentration of >10 nM and a primer concentration of 0.1 μM and 0.2 μM failed to provide the desired full-length (760 bp) product. In contrast, syntheses performed with 0.4 μM of primer all successfully produced the target 760 bp DNA. Of the four samples, sample 2-3 with an oligonucleotide concentration of 10 nM and a primer concentration of 0.4 μM produced the most full-length product, even though samples 3-3 (15 nM, 0.4 μM) and 4-3 (25 nM, 0.4 μM) have more full-length DNA generated initially from the PCA step. Based on these findings, an oligo-nucleotide concentration of 10 nM and a primer concentration of 0.4 μM were selected for gene synthesis on a microfluidic device.

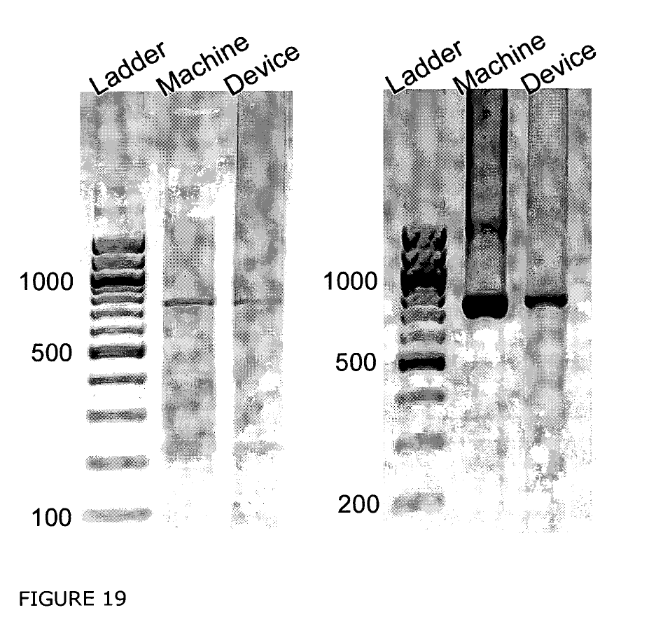

With the optimized oligonucleotide and primer concentrations, GFPuv (760 bp) was successfully synthesized from a pool of short oligonucleotids (40 bases) by using either one-step (single-chamber chip) or two-step microfluidic devices. Strong, dominant band of the desired products were obtained in the gel images (FIG. 19). The visually estimated yields of microfluidic devices were ˜50% of the controls performed in PCR tubes with a commercial thermal cycler. These were limited by the dead volume (2.87 μL) in the channels between the PCR chamber (7 μL) and the valves. The oligonucleotides mixture within the dead volume did not assemble, but contributed to ˜30% of the eluted solution. The gel results also demonstrated that parylene was compatible with PCR reaction mixture, and effectively blocked the reagents against evaporation from the water vapor-permeable PDMS.

Compared to the one-step process, the two-step process generated much more full-length product from the same amount of initial oligonucleotides. In the one-step process, the assembly and amplification were conducted simultaneously, which competed for the fixed amount oligonucleotides and monomers (dNTPs), and rendered intermediary products with lower molecular weights (FIG. 19a). The process competition was minimized in the two-step process, resulting in more full-length product. The two-step process was reported to be more reliable than the one-step process, which sometimes failed to generate full-length DNA (Gao et al, Nucleic Acids Res., 2003, 31, e143; Xiong et al, Nucleic Acids Res., 2004, 32, e98). Gene synthesis with the two-step process also allowed for different annealing temperatures to optimize the assembly and amplification processes separately.

The assembled sequence was identified by DNA sequencing. Synthesized products from the microfluidic devices and PCR tubes were cloned directly without further purification using PCR®2.1-TOPO® cloning vector (Invitrogen). Full-length target along with intermediary products were all cloned to reflect the real composition of the synthesized products.

Table 1 shows the sequencing results. The error rates per kilobase (kb) calculated from full-length clones were 3.45 in device and 4.36 in PCR tube for the one-step process, and 4.01 in device and 4.10 in PCR tube for the two-step process. These values were within the range of the error rates reported (1.8-6 per kb) (Tian et al, Nature, 2004, 432, 1050; Xiong et al, Nucleic Acids Res., 2004, 32, e98; Hoover and J. Lubkowski, Nucleic Acids Res., 2002, 30, e43; Withers-Martinez et al, Protein Engr., 1999, 12, 1113).

Most errors (>85%) were associated with single-base insertion, deletion and mutation. The indifference in error rates implied that they were independent of the synthesis methods (device versus PCR tube) and processes (one-step versus two-step). Hoover and J. Lubkowski, Nucleic Acids Res., 2002, 30, e43 and Tian et al, Nature, 2004, 432, 1050 pointed out that the greatest errors were attributed to the quality of synthetic oligonucleotides, not from the fidelity of polymerase enzyme. Oligonucleotides were chemically synthesized base-by-base with a step yield of ˜98.5% (Hecker and Rill, BioTechniques, 1998, 24, 256). The overall yield of full-length oligonucleotides decreased as the oligo-nucleotide length increased. For example, only 54.6% of oligo-nucleotides was full-length in a targeted 40 base-long synthesis product.

The building blocks of synthetic oligonucleotides containing both perfect match sequence and impurities with mismatch (single base and multiple bases) could all have participated in the PCR process and generated products of incorrect sequence. In contrast, the DNA polymerase has a replication fidelity of ˜10⁻⁶ base/duplication (Cline et al, Nucleic Acids Res., 1996, 24, 3546), which was 3-4 orders lower than the error rate of synthetic gene products. Performing gene synthesis in a microfluidic device might not improve the accuracy of synthesis products as demonstrated by Kong et al (Nucleic Acids Res., 2007, 35(8):e61, e-pub Apr. 2, 2007) in microPCR one-step gene synthesis. However, it would reduce the handling time and reagents costs, and eliminate human process factors.

PCR product cloning and DNA sequencing were required to ensure that an accurate synthesis product was obtained. These processes involved substantial laboratory efforts. To obtain an error-free gene, many randomly selected clones were sequenced (Binkowski et al, Nucleic Acids Res., 2005, 33, e55; Carr et al, Nucleic Acids Res., 2004, 32, e162), which might contain either undesired truncated DNAs or the desired full-length DNA. The greater full-length yield of the two-step process increased the possibility in obtaining effective full-length clones, and in achieving an error-free gene. About three out of four clones (35/47 in PCR tube) produced by the two-step process contained full-length products, which was greater than that produced in the one-step process (about one out of three clones (16/47 in PCR tube)) (Table 1). Therefore, the two-step process would be preferred in minimizing the number of colony sequencing required to obtain an error-free gene, and the effort of cloning and DNA sequencing, especially for long DNAs.

### Thermally Enhanced Solid-Phase PCR Purification

For applications such as cell-free protein synthesis (Mei et al, Anal. Chem., 2006, 78, 7659; Noireaux and Libchaber, Proc. Natl. Acad. Sci. USA, 2004, 101, 17669) (which directly use synthetic genes for protein expression) and integration of enzymatic error filtering methods (Binkowski et al, Nucleic Acids Res., 2005, 33, e55; Carr, et al, Nucleic Acids Res., 2004, 32, e162; Fuhrmann et al, Nucleic Acids Res., 2005, 33, e58; Brown et al, Biochem. J., 2001, 354, 62725), on chip to reduce the error rate of synthesized products, a solid-phase buffer exchange process was integrated with the two-step microfluidic device utilizing magnetic beads based PCR purification method (ChargeSwitch PCR clean-up Kits, Invitrogen). This process was intended to purify the assembled product from short primers and dNTPs, and to prepare the buffer solution for downstream application. Silica-coated magnetic beads could help simplify the device integration (Liu et al, Anal. Chem., 2004, 76, 1824; Cho et al, Lab Chip, 2007, 7, 565) as compared to other nucleic acid extraction methods reported by Jemere et al, Electrophoresis, 2002, 23, 3537; West et al, Sens. Actuators B, 2007, 126, 664; and Breadmore et al, Anal. Chem., 2003, 75, 1880.

ChargeSwitch utilized the same approach as other reported methods (West et al, Sens. Actuators B, 2007, 126, 664; Breadmore et al, Anal. Chem., 2003, 75, 1880). DNA was first adsorbed onto the silica surface under high ionic strength conditions. The unbound impurities were washed away, and then the adsorbed DNA was released into solution with a higher pH (10 mM of Tris-HCI, pH 8.5). The ChargeSwitch Kit was first optimized in standard PCR tubes using 100 bp DNA ladder with a known DNA quantity (1.19 μg) as the control following the approach and protocol suggested by manufacturer.

The reagents volume was modified to match the design of microfluidic device. After the baseline protocol was established using PCR tube and 100 bp ladder, the procedure was applied to microfluidic device for 100 bp DNA ladder and PCR synthesized product. The total amount of 100 bp DNA ladder (1.19 μg) or PCR product (1.98 μg) was less than the binding capability of the ChargeSwitch beads loaded. Based on the manufacturer's protocol, the ChargeSwitch beads would bind double-stranded DNAs with lengths of >90 bp; thus, 100 bp DNA ladder was selected as the control. The DNA extraction included three steps—DNA capture, impurities wash, and DNA elution. The DNA elution conditions (time and temperature) were investigated to increase the extraction efficiency.

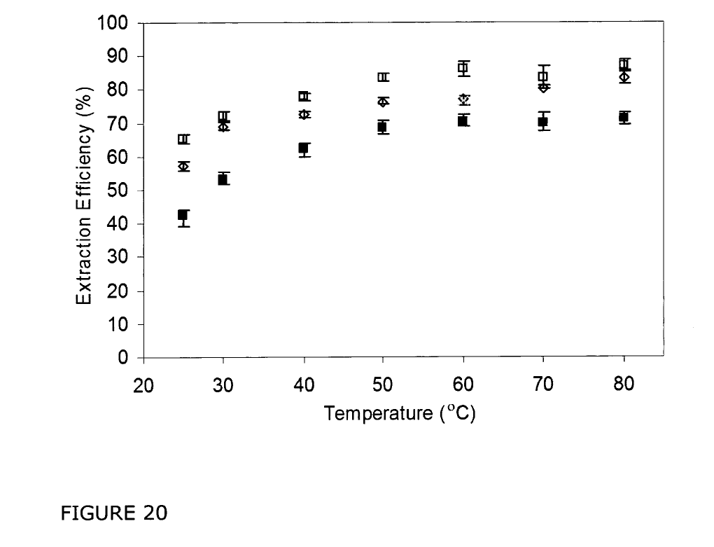

The extraction efficiency, defined as the percentage of DNA captured and released, was shown in FIG. 20. The quantities of the original and eluted DNA samples were determined by a UV-Vis spectrophotometer. Every measurement was repeated three times. The average extraction efficiency of 100 bp DNA ladder was 65.4% in PCR tube and 42.2% in microfludic device, eluted at 25° C. for 3 min in 7 μL of Tris-HCl buffer (10 mM of Tris-HCI, pH 8.5). When the adsorbed DNA was subjected to increasing elution temperature, the release of bound DNA was enhanced. The extraction efficiency increased effectively to 86% in PCR tube and 70% in microfluidic device when incubated at 60° C. for 3 min. Further increase in temperature did not improve the extraction efficiency. The extraction efficiency was slightly improved (<10%) with increasing incubation time (2 min to 3 min). The thermally enhanced DNA elution could be due to either the temperature effect of pH variation in Tris-HCl buffer or the increased thermal momentum of bound DNA (Cline et al, Nucleic Acids Res., 1996, 24, 3546).

For PCR synthetic product, the extraction efficiencies were 76.6% (+3.38%, −5.66%) in PCR tube and 61.3% (+3.51%, −2.56%) in microfluidic device when incubated at 60° C. for 3 min. These efficiencies were lower than the 86% and 70.1% achieved for 100 bp DNA ladder, respectively. The differences could be due to the short primers and monomers (dNTPs), which also absorbed UV light at a wavelength of 260 nm like dsDNA. The drop in extraction efficiency indicated that the impurities (short primers and monomers (dNTPs)) in the PCR product were removed. This thermally enhanced DNA extraction was simple, and provided an extraction efficiency that was close to that achieved with sol-gel derived silica particles (65%) (Breadmore et al, Anal. Chem., 2003, 75, 1880) and monolithic sol-gel microchip (85%) (Wu et al, Anal. Chem., 2006, 78, 5704), and densely packed microfabricated silicon structure (75%) (West et al, Sens. Actuators B, 2007, 126, 664). It could be easily integrated with most microfluidic devices without extra fabrication steps or modification, and allowed successful extraction of microgram quantities of DNA in 7 μL of elution buffer in <20 min. The high loading capacity (micrograms) was particularly desirable for extracting PCR-synthesized products. Most solid-phase DNA extraction chips (West et al, Sens. Actuators B, 2007, 126, 664; Breadmore et al, Anal. Chem., 2003, 75, 1880; Wu et al, Anal. Chem., 2006, 78, 5704; Samper et al, Sens. Actuators A, 2007, 139, 139) were designed for DNA purification from biological samples, having a binding capacity of nanograms only. The short heat shock (3 min) effectively increased the extraction efficiency from 42.2% (25° C.) to 70% (60° C.) in the microfluidic device.

The present invention is not to be limited in scope by the specific embodiments described herein. Various modifications of the invention in addition to those described herein will become apparent to those skilled in the art from the foregoing description and accompanying figures. Such modifications are intended to fall within the scope of the claims.

The present invention is directed to each individual feature, system, material and/or method described herein. In addition, any combination of two or more such features, systems, materials and/or methods, provided that such features, systems, materials and/or methods are not mutually inconsistent, is included within the scope of the present invention.

In the specification and claims, all transitional phrases or phrases of inclusion, such as “comprising,” “including,” “carrying,” “having,” “containing,” “composed of,” “made of,” “formed of,” “involving” and the like shall be interpreted to be open-ended, i.e. to mean “including but not limited to” and, therefore, encompassing the items listed thereafter and equivalents thereof as well as additional items. Only the transitional phrases or phrases of inclusion “consisting of” and “consisting essentially of” are to be interpreted as closed or semi-closed phrases, respectively. The indefinite articles “a” and “an,” as used herein in the specification and in the claims, unless clearly indicated to the contrary, should be understood to mean “at least one.” The expression “A or B”, unless clearly indicated to the contrary, should be understood to mean “A or B or both”.

Various publications are cited herein, the disclosures of which are incorporated by reference in their entirety or in pertinent part, as is understood from the context of the publication being cited. In cases where the present specification and a document incorporated by reference and/or referred to herein include conflicting disclosure, and/or inconsistent use of terminology, and/or the incorporated/referenced documents use or define terms differently than they are used or defined in the present specification, the present specification shall control.

TABLE 1
Errors and efficiencies in the synthesis of GFPuv
using one-step and two-step processes in the microfluidic
device vs. standard PCR tube (machine).

| Error type | One-step | | Two-step | |
|---|---|---|---|---|
| Single deletion | 19 | 35 | 45 | 73 |
| Multiple deletion | 6 | 6 | 5 | 9 |
| Insertion | 5 | 2 | 1 | 5 |
| Mutation | 12 | 10 | 10 | 22 |
| Total error | 42 | 53 | 61 | 109 |
| Bases sequenced | 12160 | 12160 | 15200 | 26600 |
| Error rate (per 1 kb) | 3.45 | 4.36 | 4.01 | 4.10 |
| Truncated clones | 38/54 | 31/47 | 23/43 | 12/47 |
| Full-length clones | 16/54 | 16/47 | 20/43 | 35/47 |

TABLE 2
Oligonucleotides set for the 760 by GFPuv segment

| | SEQ | Oligonucleotide sequence | Tₘ | Overlap | Length |
|---|---|---|---|---|---|
| Label | ID NO | (5′ to 3′) | (° C.) | (bp) | (nt) |
| F0 | 1 | AGAGGATCCCCGGGTACCGGTAGAAAAAATGAGTAAAGGA | 44.5 | 20 | 40 |
| R0 | 2 | ACTCCAGTGAAAAGTTCTTCTCCTTTACTCATTTTTTCTA | 50.7 | 20 | 40 |
| F1 | 3 | GAAGAACTTTTCACTGGAGTTGTCCCAATTCTTGTTGAAT | 50.5 | 20 | 40 |
| R1 | 4 | CCCGTTAACATCACCATCTAATTCAACAAGAATTGGGACA | 52.2 | 20 | 40 |
| F2 | 5 | TAGATGGTGATGTTAACGGGCACAAATTTTCTGTCAGTGG | 50.7 | 20 | 40 |
| R2 | 6 | TTGCATCACCTTCACCCTCTCCACTGACAGAAAATTTGTG | 56.6 | 20 | 40 |
| F3 | 7 | AGAGGGTGAAGGTGATGCAACATACGGAAAACTTACCCTT | 50.5 | 20 | 40 |
| R3 | 8 | CCAGTAGTGCAAATAAATTTAAGGGTAAGTTTTCCGTATG | 47.1 | 20 | 40 |
| F4 | 9 | AAATTTATTTGCACTACTGGAAAACTACCTGTTCCATGGC | 53.6 | 20 | 40 |
| R4 | 10 | GAAAGTAGTGACAAGTGTTGGCCATGGAACAGGTAGTTTT | 49.8 | 20 | 40 |
| F5 | 11 | CAACACTTGTCACTACTTTCTCTTATGGTGTTCAATGCTT | 50.4 | 20 | 40 |
| R5 | 12 | TATGATCCGGATAACGGGAAAAGCATTGAACACCATAAGA | 53.2 | 20 | 40 |
| F6 | 13 | TTCCCGTTATCCGGATCATATGAAACGGCATGACTTTTTC | 52.8 | 20 | 40 |
| R6 | 14 | CCTTCGGGCATGGCACTCTTGAAAAAGTCATGCCGTTTCA | 60.5 | 20 | 40 |
| F7 | 15 | AAGAGTGCCATGCCCGAAGGTTATGTACAGGAACGCACTA | 51.5 | 20 | 40 |
| R7 | 16 | CCCGTCATCTTTGAAAGATATAGTGCGTTCCTGTACATAA | 50.4 | 20 | 40 |
| F8 | 17 | TATCTTTCAAAGATGACGGGAACTACAAGACGCGTGCTGA | 57.7 | 20 | 40 |
| R8 | 18 | TATCACCTTCAAACTTGACTTCAGCACGCGTCTTGTAGTT | 49.3 | 20 | 40 |
| F9 | 19 | AGTCAAGTTTGAAGGTGATACCCTTGTTAATCGTATCGAG | 51.1 | 20 | 40 |
| R9 | 20 | TTAAAATCAATACCTTTTAACTCGATACGATTAACAAGGG | 40.6 | 20 | 40 |
| F10 | 21 | TTAAAAGGTATTGATTTTAAAGAAGATGGAAACATTCTCG | 50.7 | 20 | 40 |
| R10 | 22 | GTTGTACTCGAGTTTGTGTCCGAGAATGTTTCCATCTTCT | 52.1 | 20 | 40 |
| F11 | 23 | GACACAAACTCGAGTACAACTATAACTCACACAATGTATA | 43.6 | 20 | 40 |
| R11 | 24 | TTTGTTTGTCTGCCGTGATGTATACATTGTGTGAGTTATA | 54.9 | 20 | 40 |
| F12 | 25 | CATCACGGCAGACAAACAAAAGAATGGAATCAAAGCTAAC | 48.7 | 20 | 40 |
| R12 | 26 | ATGTTGTGGCGAATTTTGAAGTTAGCTTTGATTCCATTCT | 52.3 | 20 | 40 |
| F13 | 27 | TTCAAAATTCGCCACAACATTGAAGATGGAAGCGTTCAAC | 54.1 | 20 | 40 |
| R13 | 28 | TTGTTGATAATGGTCTGCTAGTTGAACGCTTCCATCTTCA | 49.5 | 20 | 40 |
| F14 | 29 | TAGCAGACCATTATCAACAAAATACTCCAATTGGCGATGG | 53.5 | 20 | 40 |
| R14 | 30 | TGTCTGGTAAAAGGACAGGGCCATCGCCAATTGGAGTATT | 54.4 | 20 | 40 |
| F15 | 31 | CCCTGTCCTTTTACCAGACAACCATTACCTGTCGACACAA | 54.5 | 20 | 40 |
| R15 | 32 | GGATCTTTCGAAAGGGCAGATTGTGTCGACAGGTAATGGT | 55.3 | 20 | 40 |
| F16 | 33 | TCTGCCCTTTCGAAAGATCCCAACGAAAAGCGTGACCACA | 57.4 | 20 | 40 |
| R16 | 34 | TACAAACTCAAGAAGGACCATGTGGTCACGCTTTTCGTTG | 51.4 | 20 | 40 |
| F17 | 35 | TGGTCCTTCTTGAGTTTGTAACTGCTGCTGGGATTACACA | 57.0 | 20 | 40 |
| R17 | 36 | TGTAGAGCTCATCCATGCCATGTGTAATCCCAGCAGCAGT | 56.2 | 20 | 40 |
| F18 | 37 | TGGCATGGATGAGCTCTACAAATAATGAATTCCAACTGAG | 46.2 | 20 | 40 |
| R18 | 38 | CTCAGTTGGAATTCATTATT | | | 20 |
| F_Primer | 39 | AGAGGATCCCCGGGTACCGG | 62.5 | | 20 |
| R_Primer | 40 | CTCAGTTGGAATTCATTATT | 46.2 | | 20 |

1. A method for synthesizing double-stranded DNA in a microfluidic device, the device comprising a PCR-assembly (PCA) chamber in controllable fluid communication with a polymerase chain reaction (PCR) chamber, the method comprising the steps of:
(a) applying a time-varying thermal field to the PCA chamber containing a plurality of different oligonucleotides and polymerase, wherein each oligonucleotide has partial base complementarity with at least one other oligonucleotide, thereby assembling the oligonucleotides into templates for PCR in the absence of terminal PCR primers;
(b) loading the templates produced in step (a) into the PCR chamber in the presence of a PCR precursor mix comprising the terminal PCR primers, dNTPs and polymerase; and
(c) applying a time-varying thermal field to the PCR chamber, thereby obtaining a PCR product mixture comprising the double-stranded DNA.

2. The method of claim 1 wherein the device further comprises a purification chamber in controllable fluid communication with the PCR chamber, the method further comprising the step of:
(d) loading the PCR product mixture into the purification chamber to immobilize the double-stranded DNA, thereby separating the double-stranded DNA from free dNTPs, primers and unpolymerized oligonucleotides.

3. The method of claim 2 wherein the double-stranded DNA is immobilized on magnetic beads.

4. The method of claim 3 further comprising the step of extracting the double-stranded DNA from the magnetic beads by subjecting the bead-immobilized DNA to heatshock conditions of 60° C. for 3 minutes.

5. The method of claim 2 wherein the device further comprises an error filtration chamber in controllable fluid communication with the purification chamber, the method further comprising the step of:
(e) loading the double-stranded DNA produced in step (d) into the error filter chamber to remove double-stranded DNA that contain base-pair mismatches.

6. The method of claim 1 wherein the device further comprises a purification chamber in controllable fluid communication with the PCA chamber, the method further comprising the step of:
(d) loading the templates produced in step (a) into the purification chamber to immobilize the templates, thereby separating the templates from free dNTPs and unpolymerized oligonucleotides; and then proceeding to step (b).

7. The method of claim 6 wherein the templates are immobilized on magnetic beads.

8. The method of claim 7 further comprising the step of extracting the templates from the magnetic beads by subjecting the bead-immobilized templates to heatshock conditions of 60° C. for 3 minutes.

9. The method of claim 6 wherein the device further comprises an error filtration chamber in controllable fluid communication with the purification chamber, the method further comprising the step of:
(e) loading the template produced in step (d) into the error filter chamber to remove templates that contain base-pair mismatches; and then proceeding to step (b).

10. The method of claim 1 wherein the device further comprises a micro-mixer, the method further comprising the step of:
in step (b), mixing the PCR precursor mix with the templates produced in step (a); and/or
in step (d), mixing the PCR product mixture with DNA-adsorbing solid phase media.

11. A method for synthesizing double-stranded DNA in a microfluidic device, the device comprising a synthesis chamber in controllable fluid communication with a purification chamber, the method comprising the steps of:
(a) applying a time-varying thermal field to the synthesis chamber containing terminal PCR primers, polymerase, dNTPs and a plurality of different oligonucleotides wherein each oligonucleotide has partial base complementarity with at least one other oligonucleotide, thereby obtaining a PCR product mixture comprising the double-stranded DNA; and
(b) loading the PCR product mixture into the purification chamber to immobilize the double-stranded DNA, thereby separating the double-stranded DNA from free dNTPs, primers and unpolymerized oligonucleotides.

12. The method of claim 11 wherein the double-stranded DNA in step (b) is immobilized on magnetic beads.

13. The method of claim 12 further comprising the step of extracting the double-stranded DNA from the magnetic beads by subjecting the bead-immobilized DNA to heatshock conditions of 60° C. for 3 minutes.

14. The method of claim 11 wherein the device further comprises an error filtration chamber in controllable fluid communication with the purification chamber, the method further comprising the step of:
(c) loading the double-stranded DNA produced in step (b) into the error filter chamber to remove double-stranded DNA that contain base-pair mismatches.

15. The method of claim 11 wherein the device further comprises a micro-mixer, the method further comprising the step of mixing the PCR product mixture in step (b) with DNA-adsorbing solid phase media.

16. The method of claim 1 wherein the device is operably linked to a fluid-flow actuator.

17. The method of claim 16 wherein the fluid-flow actuator is a pump or a centrifuge.

18. The method of claim 1 wherein the chambers are in controllable fluid communication with one another via channels comprising valves.

19. The method of claim 18 wherein the valves are responsive to temperature changes and wherein the valves that control sealing of the PCR chamber are able to withstand at least 6.8 psi of pressure.

20. The method of claim 1 wherein the device is operably linked to a heating element, a cooling element, a temperature-sensor, and a temperature controller.


SEQUENCE LISTING (40 sequences)

SEQ ID NO: 1 - DNA, 40 bp, Artificial Sequence
  synthetic oligonucleotide primer
agaggatccccgggtaccggtagaaaaaatgagtaaagga

SEQ ID NO: 2 - DNA, 40 bp, Artificial Sequence
  synthetic oligonucleotide primer
actccagtgaaaagttcttctcctttactcattttttcta

SEQ ID NO: 3 - DNA, 40 bp, Artificial Sequence
  synthetic oligonucleotide primer
gaagaacttttcactggagttgtcccaattcttgttgaat

SEQ ID NO: 4 - DNA, 40 bp, Artificial Sequence
  synthetic oligonucleotide primer
cccgttaacatcaccatctaattcaacaagaattgggaca

SEQ ID NO: 5 - DNA, 40 bp, Artificial Sequence
  synthetic oligonucleotide primer
tagatggtgatgttaacgggcacaaattttctgtcagtgg

SEQ ID NO: 6 - DNA, 40 bp, Artificial Sequence
  synthetic oligonucleotide primer
ttgcatcaccttcaccctctccactgacagaaaatttgtg

SEQ ID NO: 7 - DNA, 40 bp, Artificial Sequence
  synthetic oligonucleotide primer
agagggtgaaggtgatgcaacatacggaaaacttaccctt

SEQ ID NO: 8 - DNA, 40 bp, Artificial Sequence
  synthetic oligonucleotide primer
ccagtagtgcaaataaatttaagggtaagttttccgtatg

SEQ ID NO: 9 - DNA, 40 bp, Artificial Sequence
  synthetic oligonucleotide primer
aaatttatttgcactactggaaaactacctgttccatggc

SEQ ID NO: 10 - DNA, 40 bp, Artificial Sequence
  synthetic oligonucleotide primer
gaaagtagtgacaagtgttggccatggaacaggtagtttt

SEQ ID NO: 11 - DNA, 40 bp, Artificial Sequence
  synthetic oligonucleotide primer
caacacttgtcactactttctcttatggtgttcaatgctt

SEQ ID NO: 12 - DNA, 40 bp, Artificial Sequence
  synthetic oligonucleotide primer
tatgatccggataacgggaaaagcattgaacaccataaga

SEQ ID NO: 13 - DNA, 40 bp, Artificial Sequence
  synthetic oligonucleotide primer
ttcccgttatccggatcatatgaaacggcatgactttttc

SEQ ID NO: 14 - DNA, 40 bp, Artificial Sequence
  synthetic oligonucleotide primer
ccttcgggcatggcactcttgaaaaagtcatgccgtttca

SEQ ID NO: 15 - DNA, 40 bp, Artificial Sequence
  synthetic oligonucleotide primer
aagagtgccatgcccgaaggttatgtacaggaacgcacta

SEQ ID NO: 16 - DNA, 40 bp, Artificial Sequence
  synthetic oligonucleotide primer
cccgtcatctttgaaagatatagtgcgttcctgtacataa

SEQ ID NO: 17 - DNA, 40 bp, Artificial Sequence
  synthetic oligonucleotide primer
tatctttcaaagatgacgggaactacaagacgcgtgctga

SEQ ID NO: 18 - DNA, 40 bp, Artificial Sequence
  synthetic oligonucleotide primer
tatcaccttcaaacttgacttcagcacgcgtcttgtagtt

SEQ ID NO: 19 - DNA, 40 bp, Artificial Sequence
  synthetic oligonucleotide primer
agtcaagtttgaaggtgatacccttgttaatcgtatcgag

SEQ ID NO: 20 - DNA, 40 bp, Artificial Sequence
  synthetic oligonucleotide primer
ttaaaatcaataccttttaactcgatacgattaacaaggg

SEQ ID NO: 21 - DNA, 40 bp, Artificial Sequence
  synthetic oligonucleotide primer
ttaaaaggtattgattttaaagaagatggaaacattctcg

SEQ ID NO: 22 - DNA, 40 bp, Artificial Sequence
  synthetic oligonucleotide primer
gttgtactcgagtttgtgtccgagaatgtttccatcttct

SEQ ID NO: 23 - DNA, 40 bp, Artificial Sequence
  synthetic oligonucleotide primer
gacacaaactcgagtacaactataactcacacaatgtata

SEQ ID NO: 24 - DNA, 40 bp, Artificial Sequence
  synthetic oligonucleotide primer
tttgtttgtctgccgtgatgtatacattgtgtgagttata

SEQ ID NO: 25 - DNA, 40 bp, Artificial Sequence
  synthetic oligonucleotide primer
catcacggcagacaaacaaaagaatggaatcaaagctaac

SEQ ID NO: 26 - DNA, 40 bp, Artificial Sequence
  synthetic oligonucleotide primer
atgttgtggcgaattttgaagttagctttgattccattct

SEQ ID NO: 27 - DNA, 40 bp, Artificial Sequence
  synthetic oligonucleotide primer
ttcaaaattcgccacaacattgaagatggaagcgttcaac

SEQ ID NO: 28 - DNA, 40 bp, Artificial Sequence
  synthetic oligonucleotide primer
ttgttgataatggtctgctagttgaacgcttccatcttca

SEQ ID NO: 29 - DNA, 40 bp, Artificial Sequence
  synthetic oligonucleotide primer
tagcagaccattatcaacaaaatactccaattggcgatgg

SEQ ID NO: 30 - DNA, 40 bp, Artificial Sequence
  synthetic oligonucleotide primer
tgtctggtaaaaggacagggccatcgccaattggagtatt

SEQ ID NO: 31 - DNA, 40 bp, Artificial Sequence
  synthetic oligonucleotide primer
ccctgtccttttaccagacaaccattacctgtcgacacaa

SEQ ID NO: 32 - DNA, 40 bp, Artificial Sequence
  synthetic oligonucleotide primer
ggatctttcgaaagggcagattgtgtcgacaggtaatggt

SEQ ID NO: 33 - DNA, 40 bp, Artificial Sequence
  synthetic oligonucleotide primer
tctgccctttcgaaagatcccaacgaaaagcgtgaccaca

SEQ ID NO: 34 - DNA, 40 bp, Artificial Sequence
  synthetic oligonucleotide primer
tacaaactcaagaaggaccatgtggtcacgcttttcgttg

SEQ ID NO: 35 - DNA, 40 bp, Artificial Sequence
  synthetic oligonucleotide primer
tggtccttcttgagtttgtaactgctgctgggattacaca

SEQ ID NO: 36 - DNA, 40 bp, Artificial Sequence
  synthetic oligonucleotide primer
tgtagagctcatccatgccatgtgtaatcccagcagcagt

SEQ ID NO: 37 - DNA, 40 bp, Artificial Sequence
  synthetic oligonucleotide primer
tggcatggatgagctctacaaataatgaattccaactgag

SEQ ID NO: 38 - DNA, 20 bp, Artificial Sequence
  synthetic oligonucleotide primer
ctcagttggaattcattatt

SEQ ID NO: 39 - DNA, 20 bp, Artificial Sequence
  synthetic oligonucleotide primer
agaggatccccgggtaccgg

SEQ ID NO: 40 - DNA, 20 bp, Artificial Sequence
  synthetic oligonucleotide primer
ctcagttggaattcattatt